(19) United States
(12) Patent Application Publication
(10) Pub. No.: US 2023/0117397 A1
(43) Pub. Date: Apr. 20, 2023

(54) PIRFENIDONE FOR CORONAVIRUS TREATMENT

(71) Applicant: Excalibur Pharmaceuticals, Inc., New York, NY (US)

(72) Inventors: Estuardo Aguilar-Cordova, Mexico City (MX); Laura Aguilar, Mexico City (MX); José Agustín Rogelio Magaña Castro, Mexico City (MX)

(73) Assignee: Excalibur Pharmaceuticals, Inc., New York, NY (US)

(21) Appl. No.: 17/918,791

(22) PCT Filed: Apr. 14, 2021
(86) PCT No.: PCT/US2021/027335

§ 371 Date: Oct. 13, 2022

Related U.S. Application Data
(60) Provisional application No. 63/010,009, filed on Apr. 14, 2020.

(51) Int. Cl. A61K 31/4418; A61P 31/14; A61P 29/00; A61K 9/20

(52) U.S. Cl. CPC A61K 31/4418; A61P 31/14; A61P 29/00; A61K 9/2054; A61K 9/2018

(57) ABSTRACT

Provided herein are methods and uses involving pirfenidone pharmaceutical compositions (e.g., extended-release pirfenidone pills (e.g., tablets)) in treating COVID-19, treating and/or preventing viremia and/or viremia-induced disease caused by a virus, and/or an infection with a virus (e.g., DNA virus, RNA virus, for example, coronavirus (e.g., severe acute respiratory syndrome coronavims-2 (SARS-CoV-2) and variants thereof, severe acute respiratory syndrome-associated coronavirus (SARS-CoV), Middle East respiratory syndrome coronavirus (MERS-CoV)), in a subject (e.g., subject with COVID-19). Provided are methods of reducing elevated levels of one or more markers of inflammation (e.g., CRP, ferritin, LDH), inhibiting an inflammatory response (e.g., virus-induced) and/or inhibiting a cascade by one or more pro-inflammatory cytokines (e.g., TNF-α, IEN-γ, IL-Iβ, IL-6, IL-12) and/or in a subject (e.g., a subject with COVID-19 optionally having liver contraindications for pirfenidone).

### RELATED APPLICATIONS

This application claims priority under 35 U.S.C. § 119(e) to U.S. Provisional Application, U.S. Ser. No. 63/010,009, filed Apr. 14, 2020, which is incorporated herein by reference.

### BACKGROUND OF THE INVENTION

Coronavirus disease 2019 (COVID-19) is an emerging infectious disease caused by infection with severe acute respiratory syndrome coronavirus-2 (SARS-CoV-2). This highly contagious viral infection causes significant morbidity and mortality due to acute respiratory distress syndrome (ARDS) and multi-organ failure. The ongoing COVID-19 pandemic is overwhelming the health care system with large numbers of patients requiring hospitalization, intensive care unit (ICU) admission and ventilator support. Hospitalized patients exhibit exceedingly high levels of serum markers of acute inflammation including CRP, ferritin, and LDH. Accumulating evidence suggests that high levels of inflammatory mediators may be associated with worse outcomes. The disease has two stages, the first an infectious viremic stage and a second inflammatory stage. Both stages need to be addressed. It is not clear what makes some patients enter the inflammatory stage while others do not. In addition, persistent lung inflammation, even if resolved, may lead to long-term sequela such as lung fibrosis.

### SUMMARY OF THE INVENTION

Pirfenidone is an anti-inflammatory and anti-fibrotic drug that is approved for the treatment of idiopathic pulmonary fibrosis. Pirfenidone XR (Kitoscell® LP) is an extended release pirfenidone formulation approved in Mexico with ongoing development in other countries. The profile of cytokines impacted by Pirfenidone XR (Kitoscell® LP) are consistent with those that are abnormal in patients with COVID-19, and which may be the root cause of the inflammatory stage of the disease. Thus, it is proposed that Kitoscell® LP treatment may protect SARS-CoV-2 infected patients from clinical deterioration, including ARDS and other inflammation-related morbidities, decrease the rate of patients requiring ICU or ventilator support, and thus overall improve the outcome for COVID-19 patients.

Pirfenidone [5-methyl-1-phenyl-2-(1H)-pyridone] (shown below) is a small synthetic molecule with a molecular weight of 185.2 (FIG. 2) that has been shown to be quickly absorbed from the gastrointestinal tract, metabolized primarily by the liver cytochrome P450 enzyme CYP1A2 and secreted mostly as its non-active metabolite 5-carboxy-pirfenidone. As shown in Scheme 1 below, pirfenidone can be hydrolyzed to its non-functional metabolite.

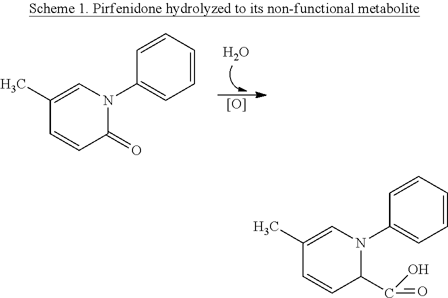

Pirfenidone has a short in-vivo half-life. After oral administration, pirfenidone is distributed throughout the body and has been shown to penetrate and modulate the inflammatory and fibrotic processes in many organ systems including lung, liver and kidney (Sun et al., 2016). Pirfenidone is mostly secreted in urine as its major metabolite 5-carboxy-pirfenidone, which seems to have limited to no biological activity. Thus, patients with impaired hepatic function or decreased CYP1A2 activity, may have an increased resident time of non-metabolized pirfenidone and those with impaired renal function may have increased non-functional metabolite exposure. However, it is presently observed that a major impediment to its therapeutic effectiveness may be the short duration of biologically active pirfenidone concentrations in the target tissues. The pirfenidone molecule is able to move through cell membranes without requiring a receptor. It reaches its maximum serum concentration within an hour of dosing and has a terminal half-life of approximately 2 hours. Using high mass resolution MALDI-FTICR-mass spectrometry imaging, Sun et al. (2016) calculated a T_{1/2} (min) of only 67, 46 and 49 minutes in lung, liver and kidney tissue respectively. Thus, the potential time for therapeutic concentrations of pirfenidone in tissue may be very short. Kitoscell® LP is an exemplary specialized sustained release (extended-release) formulation of pirfenidone.

Meanwhile, studies indicate that standard pirfenidone inhibits pro-inflammatory cytokines, such as TNF-α, IFN-γ, IL-6, IL-1β, IL-12 and others, with concomitant up-regulation of inflammation inhibitors, such as IL-10 (Cain et al., 1998; Nakazato et al., 2002; Oku et al., 2002; Guo et al., 2018; Schaefer et al., 2011). Key studies in animals have demonstrated protective effects of pirfenidone in endotoxic shock models (Cain et al., 1998; Nakazato et al., 2002; Oku et al., 2002). When pirfenidone is given before, during or after induction of endotoxin response, protective effects are consistently seen with inhibition of inflammatory cytokine levels and decreased lethality (Cain et al., 1998; Nakazato et al., 2002; Oku et al., 2002; Guo et al., 2018). The acute inflammatory response was induced by intraperitoneal (i.p.) injection of lipopolysaccharide (LPS) and D-galactosamine and pirfenidone was evaluated for protection against inflammatory response and eventual fibrosis (Oku et al., 2002). TNF-α peaked at 75 minutes and returned to baseline by 3 hours after LPS treatment; with pirfenidone pretreatment (5 minutes before LPS), TNF-α levels are suppressed by 97%. IL-12 peaked at about 3 hours and stayed upregulated for almost 6 hours; pirfenidone pre-treatment suppressed IL-12 by 84%. IFN-γ did not start rising until 90 minutes after LPS and peaked at 4.5 hours; pirfenidone inhibited IFN-γ by 91%. Conversely, pirfenidone pre-treatment enhanced IL-10, an anti-inflammatory cytokine, expression by approximately 27-fold by 3 hours. Survival improvements are also seen in this study when pirfenidone is given before or after LPS (Oku et al., 2002). The control mice all died within 24 hours of LPS administration. Pretreatment with pirfenidone 15 minutes before yielded a dose dependent protection, 100% (8/8) survived at 500 mg/kg and 50% (4/8) at 100 mg/kg. Only a partial protection was seen if pretreatment was given 3 hours before, and none if the pretreatment occurred 6 hours or more before LPS. When a single oral dose of pirfenidone was given 1 to 5 hours post LPS, survival rates are time-dependent. All (5/5) survived when pirfenidone was given 1 hour after LPS, 4/5 survived when pirfenidone was given 3 hours after LPS and only 1/5 survived when pirfenidone was given 5 hours after LPS (Oku et al., 2002).

It is presently observed that the timing of when pirfenidone is administered and present in the system is important to its effects on inflammatory cytokine interactions. TNF-α acts as a critical up-stream initiator of inflammatory cytokine cascades (Oku et al., 2002). Interruption of the synthesis of TNF-α may inhibit a subsequent cytokine cascade and its clinical sequelae. The inhibition of both TNF-α and IFN-γ seem to be post-transcriptional as addition of pirfenidone after cell stimulation with endotoxin did not change the steady state levels of TNF-α or IFN-γ RNA, but did significantly decrease the levels of secreted protein (Nakazato et al., 2002). This helps explain the rapid action of pirfenidone on these master cytokines. However, it may also explain the quick return of the cytokine levels if the transcriptional stimulant persists while the local pirfenidone concentration decreases to below therapeutic levels. The effects of this are represented with the comparison of two endotoxic shock model studies. When 200 mg/kg of pirfenidone was administered immediately after LPS, TNF-α expression was inhibited, but not if pirfenidone was administered 30 or 60 minutes before or after LPS (Cain et al., 1998). In comparison, the survival rate data previously mentioned showed that 500 mg/kg of pirfenidone was protective for death from endotoxic shock in a time dependent manner (Oku et al., 2002). However, pirfenidone was still protective when given up to 3 hours before or after LPS. The time-dependent nature of the protective effects of both prophylactic and therapeutic treatment with pirfenidone, highlight the limitations of pirfenidone's short half-life in-vivo. The prophylactic data suggests that if pirfenidone falls below therapeutic levels while the inflammatory cytokine induction persists, the TNF-α expression will quickly return. The therapeutic data suggests that if pirfenidone is administered after the TNF-α levels have increased, and the therapeutic drug levels do not significantly exceed the cytokine's half-life at the time of measurement, the TNF-α levels would not reflect an inhibition. Thus, in single insult, single dose models, pirfenidone was a potent inhibitor of pro-inflammatory cytokines and could protect the animals from their fatal consequences if concurrently present at therapeutic doses.

In the human clinical setting, most inflammatory insults to the lung are likely to be persistent, such as with a continuous viral infection. Animal models show how continual dosing of pirfenidone can protect against this harmful inflammatory response resulting from persistent irritation (Guo et al., 2018; Gu et al., 2018). Using the rat silicosis model (intratracheal instillation of a silica slurry providing a continuous irritant to the lung tissue), pirfenidone given once daily at either 50 mg/kg/day or 100 mg/kg/day lead to a significant reduction of TNF-α, IL-10 and IL-6, in a dose dependent manner (Guo et al., 2018). Furthermore, the pirfenidone treated animals had significantly decreased inflammation and resulting fibrosis, with an approximately 15% dose response difference between the 50 mg/kg/day and 100 mg/kg/day treatment groups (Guo et al., 2018).

It is currently suggested that the cytokine regulatory action of pirfenidone may be beneficial for protecting patients from the cytokine storm and septic shock that can result from an acute viral infection, such as in the case of COVID-19. It is currently observed that persistent therapeutic levels of pirfenidone are critical for sustained protection. Pirfenidone has high Cₘₐₓ and a short half-life following oral administration and gastrointestinal absorption. Drug clearance was approximately 30 ml/min/kg (Bruss et al, 2004), which leads to a half-life of less than 2 hours and therefore low tissue concentration troughs (Cₘᵢₙ), even after frequent dosing. It is presently proposed that in view of post-transcriptional inhibition of TNF-α and IFN-γ, as soon as the Cₘᵢₙ falls below the inhibitory level, the cytokine cascade may immediately begin. This means the patient may go through start and stop cycles with each dosing. A sustained-release (extended release) formulation of pirfenidone (e.g., Kitoscell® LP) may address this problem. It is presently important to identify therapeutics that address the inflammatory response to decrease the severity of the disease, reduce hospitalization and ICU admission rates, and decrease morbidity and mortality. Overall, it is presently important to develop a pirfenidone formulation that avoids the adverse effects of pirfenidone while maintaining a sustained delivery of pirfenidone, to inhibit the inflammatory response (e.g., the cascade or storm of pro-inflammatory cytokines (e.g., TNF-α, IFN-γ, IL-10, IL-6, IL-12) that causes an inflammatory response causing clinical deterioration, for example, progressive acute respiratory distress syndrome (ARDS), pulmonary edema, poor lung oxygen transfer, decompensation, a need for mechanical breathing assistance, and/or multi-organ failure in a subject in need thereof, for example, a subject with COVID-19.

This disclosure is based in part on the theory that sustained-release (extended-release) pirfenidone formulations of pirfenidone can be used to administer pirfenidone over a longer period of time, compared to the immediate-release pirfenidone formulation. In particular, given the decreased maximum drug concentration (Cₘₐₓ) levels following dosing, increased half-life (T_{1/2}) and minimum drug concentration (Cₘᵢₙ) levels following dosing, and equivalent drug exposure (AUC) of the sustained-release (extended-release) pirfenidone formulation, a sustained-release (extended-release) pirfenidone formulation may decrease potential adverse events and inhibit the cascade of pro-inflammatory cytokines (e.g., TNF-α, IFN-γ, IL-1β, IL-6, IL-12) that causes an inflammatory response causing clinical deterioration, for example, progressive acute respiratory distress syndrome (ARDS), pulmonary edema, poor lung oxygen transfer, decompensation, a need for mechanical breathing assistance, and/or multi-organ failure in a subject with COVID-19; viremia caused by severe acute respiratory syndrome coronavirus-2 (SARS-CoV-2)); and/or an infection with SARS-CoV-2. It is presently proposed that a sustained-release (extended-release) pirfenidone formulation will provide improved clinical benefits in treating COVID-19, viral infections with SARS-CoV-2, and viremia caused by SARS-CoV-2, by enhancing the prophylactic and therapeutic anti-fibrotic and anti-inflammatory benefits of pirfenidone.

Described herein are methods of using pirfenidone, e.g., formulated in pharmaceutical compositions, for example, modified-release formulations (e.g., extended-release or sustained-release pirfenidone formulations, for example, an extended-release pill or sustained-release pill (e.g., extended-release tablet or a sustained-release tablet comprising approximately 100 mg to approximately 850 mg of pirfenidone) in treating COVID-19 and/or COVID-19 disease caused by a SARS-CoV-2 variant thereof in a subject in need thereof, in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), COVID-19, treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2) and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus), all in a subject in need thereof (e.g., a subject currently or previously infected with viremia caused by a virus, or currently having or previously had COVID-19).

Described herein are methods of using pirfenidone pharmaceutical compositions (e.g., sustained-release pirfenidone formulations, for example, a sustained-release pill (e.g., a sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus), all in a subject in need thereof (e.g., a subject with COVID-19). Also described herein are methods of using pirfenidone formulations (e.g., extended-release or sustained-release pirfenidone formulations, for example, an extended-release pill or sustained-release pill (e.g., extended-release tablet or a sustained-release tablet comprising approximately 100 mg to approximately 850 mg of pirfenidone) in reducing and/or inhibiting an inflammatory response, for example, wherein the inflammatory response is caused by one or more pro-inflammatory cytokines (e.g., TNF-α, IFN-γ, IL-1β, IL-1, IL-2, IL-6, IL-12, IL-17, IL-18, chemokines, IL-7, IL-10, granulocyte-colony-stimulating factor, IP-10, monocyte chemoattractant protein-1 (MCP-1), macrophage inflammatory protein-1A (MIP-1A)), reducing elevated levels of one or more markers of inflammation (e.g., C-reactive protein, ferritin, LDH), and/or inhibiting an inflammatory cascade by one or more pro-inflammatory cytokines (e.g., TNF-α, IFN-γ, IL-1β, IL-6, IL-12), all in a subject in need thereof (e.g., a subject currently or previously infected with viremia caused by a virus, or currently having or previously had COVID-19). Also described herein are methods of using pirfenidone formulations (e.g., sustained-release pirfenidone formulations, for example, a sustained-release pill (e.g., a sustained-release tablet comprising approximately 100 mg to approximately 850 mg of pirfenidone) in reducing and/or inhibiting an inflammatory response, for example, wherein the inflammatory response is caused by one or more pro-inflammatory cytokines (e.g., TNF-α, IFN-γ, IL-1, IL-6, IL-12) and/or inhibiting a cascade by one or more pro-inflammatory cytokines (e.g., TNF-α, IFN-γ, IL-1, IL-6, IL-12), all in a subject in need thereof (e.g., a subject with COVID-19).

In one aspect, the present disclosure provides methods of treating and/or preventing viremia caused by a virus in a subject in need thereof, the method comprising administering pirfenidone to the subject. In one aspect, the present disclosure provides methods of reducing the viral load (viral burden), for example, and increasing the cycle threshold, in a subject in need thereof, the method comprising administering pirfenidone to the subject. In certain embodiments, the viremia caused by a virus is an RNA virus, for example, a coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)). In certain embodiments, the subject has COVID-19. In certain embodiments, the subject currently has COVID-19 or previously had COVID-19. In certain embodiments, the subject currently or previously was infected with viremia caused by a virus (e.g., an RNA virus, for example, SARS-CoV-2). In certain embodiments, the viremia caused by a virus is an RNA virus, for example, a coronavirus (e.g., severe acute respiratory syndrome coronavirus-2 (SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), severe acute respiratory syndrome-associated coronavirus (SARS-CoV), Middle East respiratory syndrome coronavirus (MERS-CoV)). In certain embodiments, the subject has COVID-19. In certain embodiments, the subject previously had COVID-19. In certain embodiments, the subject currently or previously was infected with viremia caused by a virus (e.g., an RNA virus, for example, SARS-CoV-2).

In one aspect, the present disclosure provides methods of treating or preventing viremia-induced disease, COVID-19, and/or COVID-19 disease caused by a SARS-CoV-2 variant thereof, in a subject in need thereof, the method comprising administering pirfenidone to the subject. In certain embodiments, the viremia-induced disease is caused by an RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)). In one aspect, the present disclosure provides methods of treating or preventing viremia-induced disease in a subject in need thereof, the method comprising administering pirfenidone to the subject. In certain embodiments, the viremia-induced disease is caused by an RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)). In certain embodiments, the viremia caused by a virus is an RNA virus, for example, a coronavirus (e.g., severe acute respiratory syndrome coronavirus-2 (SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), severe acute respiratory syndrome-associated coronavirus (SARS-CoV), Middle East respiratory syndrome coronavirus (MERS-CoV)). In certain embodiments, the subject has COVID-19. In certain embodiments, the subject previously had COVID-19. In certain embodiments, the subject currently or previously was infected with viremia caused by a virus (e.g., an RNA virus, for example, SARS-CoV-2).

In another aspect, the present disclosure provides methods of treating and/or preventing an infection with a virus in a subject in need thereof, the method comprising administering pirfenidone to the subject.

In certain embodiments, the infection with a virus is with an RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)). In certain embodiments, the viremia caused by a virus is with an RNA virus, for example, a coronavirus (e.g., severe acute respiratory syndrome coronavirus-2 (SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), severe acute respiratory syndrome-associated coronavirus (SARS-CoV), Middle East respiratory syndrome coronavirus (MERS-CoV)). In certain embodiments, the infection with a virus is a DNA virus. In certain embodiments, the subject has COVID-19. In certain embodiments, the subject previously had COVID-19. In certain embodiments, the subject currently or previously was infected with viremia caused by a virus (e.g., an RNA virus, for example, SARS-CoV-2).

In another aspect, the present disclosure provides methods of inhibiting an inflammatory response by one or more pro-inflammatory cytokines (e.g., inflammatory response induced by an infection with a virus, for example, RNA virus, e.g., SARS-CoV-2) in a subject in need thereof, the method comprising administering pirfenidone to the subject. In another aspect, the present disclosure provides methods of reducing elevated levels of one or more markers of inflammation (e.g., D-dimers, C-reactive protein (CRP), ferritin, lactic acid dehydrogenase (LDH)) in a subject in need thereof (e.g., with viremia caused by a virus), In another aspect, the present disclosure provides methods of inhibiting a cascade by one or more pro-inflammatory cytokines in a subject in need thereof, the method comprising administering pirfenidone to the subject. In certain embodiments, the one or more pro-inflammatory cytokines comprise TNF-α, IFN-γ, IL-1β, IL-6, and/or IL-12. In certain embodiments, the one or more pro-inflammatory cytokines comprise, but are not limited to, TNF-α, IFN-γ, IL-1β, IL-1, IL-2, IL-6, IL-12, IL-17, IL-18, chemokines, IL-7, IL-10, granulocyte-colony-stimulating factor, IP-10, monocyte chemoattractant protein-1 (MCP-1), macrophage inflammatory protein-1A (MIP-1A)). In certain embodiments, the pirfenidone is formulated in one or more modified-release pills, including, for example, extended-release pills, sustained-release pills, controlled-release pills (e.g., extended-release tablets, sustained-release tablets, wherein each of the one or more extended-release tablets or sustained-release tablets comprises the pirfenidone (e.g., approximately 100 mg to approximately 850 mg of pirfenidone, for example, approximately 600 mg of pirfenidone) and one or more excipients (e.g., hydroxypropylmethylcellulose (HPMC), microcrystalline cellulose, silicon dioxide, and/or sodium stearyl fumarate)). In certain embodiments, the pirfenidone is formulated in one or more sustained-release pills (e.g., sustained-release tablets, wherein each of the one or more sustained-release tablets comprises the pirfenidone (e.g., approximately 100 mg to approximately 850 mg of pirfenidone, for example, approximately 600 mg of pirfenidone) and one or more excipients (e.g., hydroxypropylmethylcellulose (HPMC), microcrystalline cellulose, silicon dioxide, and/or sodium stearyl fumarate)). In certain embodiments, the pirfenidone formulation is an extended-release formulation (pill, e.g., tablet) comprising: a) approximately 100 mg to approximately 850 mg of pirfenidone;
b) approximately 100 mg to 125 mg microcrystalline cellulose;
c) approximately 50.0 mg to 100.0 mg low viscosity HPMC;
d) approximately 30.0 mg to 50.0 mg high viscosity HPMC;
e) approximately 5.0 mg to 10.0 mg silicon dioxide; and
f) approximately 5.0 mg to 10.0 mg sodium stearyl fumarate.

In certain embodiments, the pirfenidone is formulated in one or more extended-release tablets, wherein the subject has one or more liver contraindications for standard-release or extended-release pirfenidone; and each of the one or more extended-release tablets comprises:
a) approximately 100 mg to approximately 850 mg of pirfenidone;
b) approximately 100 mg to 125 mg microcrystalline cellulose;
c) approximately 50.0 mg to 100.0 mg low viscosity HPMC;
d) approximately 30.0 mg to 50.0 mg high viscosity HPMC;
e) approximately 5.0 mg to 10.0 mg silicon dioxide; and
f) approximately 5.0 mg to 10.0 mg sodium stearyl fumarate.

In certain embodiments, the pirfenidone is formulated in one or more extended-release pills, wherein each of the one or more extended-release pills comprises the pirfenidone and one or more excipients; the coronavirus is severe acute respiratory syndrome coronavirus-2 (SARS-CoV-2) and/or a SARS-CoV-2 variant thereof; and the subject has coronavirus disease 2019 (COVID-19) and/or a disease caused by a SARS-CoV-2 variant thereof. In certain embodiments, the pirfenidone formulation is an extended-release tablet of Pirfenidone XR (KitosCell® LP). In certain embodiments, the pirfenidone formulation is a sustained-release tablet of KitosCell® LP.

In another aspect, provided are uses of pirfenidone pharmaceutical compositions (e.g., modified-release formulations, including, for example, formulations that are extended-release, sustained-release, controlled-release (e.g., an extended-release or sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), Middle East respiratory syndrome coronavirus (MERS-CoV)), treating and/or preventing viremia-induced disease (e.g., caused by SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), treating and/or preventing an infection with a virus (e.g., RNA virus, for example, coronavirus (e.g., severe acute respiratory syndrome coronavirus-2 (SARS-CoV-2) and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus). In another aspect, provided are uses of pirfenidone pharmaceutical compositions (e.g., sustained-release pirfenidone formulations, for example, a sustained-release pill (e.g., a sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus).

The details of one or more embodiments of the invention are set forth herein. Other features, objects, and advantages of the invention will be apparent from the Detailed Description, Examples, Figures, and Claims.

### Definitions

Definitions of specific terms are described in more detail below.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. A non-human animal may be a transgenic animal.

The terms “administer,” “administering,” or “administration” refer to implanting, absorbing, ingesting, injecting, inhaling, or otherwise introducing an inventive compound or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who does not have and did not have a disease but is at risk of developing the disease or is at risk of regression of the disease. In certain embodiments, the subject is at a higher risk of developing the disease or at a higher risk of regression of the disease than an average healthy member of a population.

A “prophylactic” agent described herein is an agent that prevents a condition, or one or more signs or symptoms associated with the condition, or prevents its recurrence.

The terms “condition,” “disease,” and “disorder” are used interchangeably.

The term “inhibition,” “inhibiting,” “inhibit,” or “inhibitor” refer to the ability of a compound to reduce, slow, halt, or prevent activity of a particular biological process (e.g., a transcription factor) in a cell relative to vehicle.

The term “inflammatory disease” or “inflammatory condition” refers to a condition and/or disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. In certain embodiments, the inflammatory disorder is fibrosis, and the fibrosis is idiopathic pulmonary fibrosis, liver cirrhosis, cystic fibrosis, systemic sclerosis, progressive kidney disease, or cardiovascular fibrosis.

The term “coronavirus” refers to a type of common virus that infects humans, typically leading to an upper respiratory infection (URI.) At least seven different types of human coronavirus have been identified. An exemplary coronavirus is SARS-CoV-2.

The term “viremia” refers to the presence of infectious viruses in the bloodstream. “Primary viremia” refers to the spread of the virus into the blood from the initial site of infection. “Secondary viremia” refers to the second occurrence of viral replication and presence of virus in the bloodstream. “Active viremia” refers to viremia caused by the active replication of viruses after they enter the blood. “Passive viremia” refers to entry of the virus directly into the bloodstream without viral replication. For example, passive viremia could include, but is not limited to, entry of a virus from a mosquito bite.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

The terms “modified-release formulation” is an oral dosage formulation that alters the timing and/or rate of release of the active drug substance, which encompasses “sustained-release formulation,” “extended-release formulation,” and “controlled-release formulation.”

The term “extended-release formulation” refers to a formulation that provides slow delivery of the active drug substance over an extended period after administration.

The term “sustained-release formulation” refers to a formulation that continuously releases the active drug substance at a sufficiently slow and controlled rate over an extended period of time, which maintains a minimum effective concentration (MEC) of the drug in the blood at a constant level during the administration period and provides prolonged therapeutic effect after administration of one dose.

The terms “sustained-release formulation” and “extended release formulation” are used interchangeably, and refer to a drug dosage formulation with a slow, sustained release delivering a therapeutic agent at a pre-determined rate, which maintains a constant concentration of the therapeutic agent over a specific period of time.

The term “controlled-release formulation” refers to a formulation that delivers the active drug substance at a controlled rate over an extended period of time. For example, the controlled-release formulation may target a local site to elicit a specific therapeutic response.

The term “about X,” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X. For example, the referenced number is within +/−5% of value X.

Abbreviations for certain terms are described in Table A below.

TABLE A
Abbreviations for Terms

| | |
|---|---|
| AE | Adverse Event |
| ALK | Alkaline phosphatase |
| ALT | Alanine transaminase |
| ALT | Alanine aminotransferase |
| ARDS | Acute respiratory distress syndrome |
| AST | Aspartate aminotransferase |
| AUC | Area under the curve |
| CBC | Complete Blood Count |
| CDC | Centers for Disease Control |
| Cmin | Minimum concentration |
| Cmax | Maximum concentration |
| COVID-19 | Coronavirus disease 2019 |
| Cr | Creatinine |
| CRF | Case Report Forms |
| CRP | C-Reactive Protein |
| DLT | Dose limiting toxicity |
| DSMC | Data Safety Monitoring Committee |
| EC | Eligibility Checklist |
| FVC | Forced vital capacity |
| g | Gram |
| H&P | History and Physical Exam |
| ICU | Intensive care unit |
| IFN | Interferon |
| IL | Interleukin |
| IPF | Idiopathic pulmonary fibrosis |
| IR | Immediate release |
| kg | Kilogram |
| LDH | Lactate dehydrogenase |
| LPS | Lipopolysaccharide |
| MALDI-FTICR | Matrix-assisted laser desorption/ |
| | ionization-mass spectrometry |
| MedDRA | Medical Dictionary for Regulatory |
| | Activities |
| mg | Milligram |
| ml | Milliliter |
| mm | Millimeter |
| OS | Overall Survival |
| PCR | Polymerase Chain Reaction |
| PFS | Progression Free Survival |
| PIs | Principal Investigators |
| SARS-CoV-2 | Severe acute respiratory syndrome |
| | coronavirus-2 |
| SOC | Standard of Care |
| SOP | Standard Operating Procedures |
| T_{1/2} | Half-life |
| TNF | Tumor necrosis factor |
| XR | Extended release |

### BRIEF DESCRIPTION OF THE DRAWINGS

FIG. 1 is a diagram depicting treatment protocol for a randomized, placebo-controlled trial to test the safety and potential improvement in clinical outcomes for COVID-19 patients treated with KITOSCELL® LP (pirfenidone XR “extended release”) (referred to as “drug” in FIG. 1).

The data for FIGS. 2-5 is based on a population of 241 enrolled subjects (pooled, and randomized with a 2:1 ratio of active to placebo subjects). In FIGS. 2-5, data for the patients is plotted along the x-axis; each lane on the x-axis represents a patient, with the y-axis showing levels of the specific marker or metric measured at different timepoints (e.g., baseline, week 2, week 4). The reference range in each of FIGS. 2-5 is as indicated in these Figures: the reference range for C-Reactive protein (CRP) is 0-1 mg/dL, the reference range for ferritin is 23-336.2 ng/ml, and the reference range for LDH is 140-270 U/L.

FIGS. 2A-2D show the levels of C-Reactive protein (CRP) for subject patients that are hospitalized (inpatients) and for ambulatory (outpatients), at the following timepoints (baseline entry (before administration of active drug/placebo), week 2, and week 4). FIG. 2A shows the CRP levels for subjects that are inpatients, at the timepoints of baseline and week 2. FIG. 2B shows the CRP levels for subjects that are inpatients, at the timepoints of baseline, week 2, and week 4. FIG. 2C shows the CRP levels for subjects that are outpatients, at the timepoints of baseline and week 2. FIG. 2D shows the CRP levels for subjects that are outpatients, at the timepoints of baseline, week 2, and week 4.

FIGS. 3A and 3B show the cycle thresholds “cT” or “Ct,” as measured by reverse transcription polymerase chain reaction (RT-PCR) SARS-CoV-2 testing, for subject patients that are hospitalized (inpatients) and for ambulatory (outpatients), at the following timepoints (baseline entry, week 2, and/or week 4). FIG. 3A shows the cycle threshold levels for subjects that are inpatients, at the timepoints of baseline and week 2. FIG. 3B shows the cycle threshold levels for subjects that are outpatients, at the timepoints of baseline and week 2.

FIGS. 4A-4D show the levels of ferritin for subject patients that are hospitalized (inpatients) and for ambulatory (outpatients), at the following timepoints (baseline entry, week 2, and week 4). FIG. 4A shows the ferritin levels for subjects that are inpatients, at the timepoints of baseline and week 2. FIG. 4B shows the ferritin levels for subjects that are inpatients, at the timepoints of baseline, week 2, and week 4. FIG. 4C shows the ferritin levels for subjects that are outpatients, at the timepoints of baseline and week 2.

FIG. 4D shows the ferritin levels for subjects that are outpatients, at the timepoints of baseline, week 2, and week 4.

FIGS. 5A-5D shows the levels of lactic acid dehydrogenase (LDH) for subject patients that are hospitalized (inpatients) and for ambulatory (outpatients), at the following timepoints (baseline entry, week 2, and week 4). FIG. 5A shows the LDH levels for subjects that are inpatients, at the timepoints of baseline and week 2. FIG. 5B shows the ferritin levels for subjects that are inpatients, at the timepoints of baseline, week 2, and week 4. FIG. 5C shows the ferritin levels for subjects that are outpatients, at the timepoints of baseline and week 2. FIG. 5D shows the ferritin levels for subjects that are outpatients, at the timepoints of baseline, week 2, and week 4.

### DETAILED DESCRIPTION OF CERTAIN EMBODIMENTS OF THE INVENTION

The present disclosure provides methods of using pirfenidone (e.g., immediate-release formulations or modified-release formulations, including, for example, extended-release formulations, sustained-release formulations, controlled-release formulations (e.g., an extended-release or sustained-release pill (e.g., an extended-release tablet or sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), all in a subject in need thereof (e.g., a subject currently or previously infected with viremia caused by a virus, or currently having or previously had COVID-19). The present disclosure provides methods of using pirfenidone (e.g., sustained-release pirfenidone formulations, for example, a sustained-release pill (e.g., a sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), all in a subject in need thereof (e.g., a subject with COVID-19). Also described herein are methods of using pirfenidone formulations (e.g., sustained-release pirfenidone formulations, for example, a sustained-release pill (e.g., a sustained-release tablet comprising approximately 100 mg to approximately 850 mg of pirfenidone)) in reducing and/or inhibiting an inflammatory response, for example, wherein the inflammatory response is caused by one or more pro-inflammatory cytokines (e.g., TNF-α, IFN-γ, IL-1β, IL-6, IL-12), reducing elevated levels of one or more markers of inflammation (e.g., C-reactive protein, ferritin, LDH) for example, in a subject in need thereof with viremia caused by a virus, and/or inhibiting a cascade by one or more pro-inflammatory cytokines (e.g., TNF-α, IFN-γ, IL-1β, IL-6, IL-12), all in a subject in need thereof (e.g., a subject currently or previously infected with viremia caused by a virus, or currently having or previously had COVID-19).

Also disclosed are uses of pirfenidone (e.g., immediate-release formulations or modified-release formulations, including, for example, extended-release formulations, sustained-release formulations, controlled-release formulations (e.g., an extended-release or sustained-release pill (e.g., an extended-release tablet or sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), all in a subject in need thereof (e.g., a subject currently or previously infected with viremia caused by a virus, or currently having or previously had COVID-19). Also disclosed are uses of pirfenidone (e.g., sustained-release pirfenidone formulations, for example, a sustained-release pill (e.g., a sustained-release tablet comprising approximately 100 mg to approximately 850 mg of pirfenidone) in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus).

Methods of Treatment and Uses

The present disclosure provides methods of treating and/or preventing viremia caused by a virus (e.g., RNA virus, for example, a coronavirus) in a subject in need thereof, the method comprising administering pirfenidone (e.g., a sustained-release pill (e.g., a sustained-release tablet of pirfenidone)) to the subject. The present disclosure provides methods of treating COVID-19 and/or COVID-19 disease caused by a SARS-CoV-2 variant, treating and/or preventing viremia caused by a virus (e.g., RNA virus, for example, a coronavirus, influenza virus, respiratory syncytial (RSV) virus) in a subject in need thereof, the method comprising administering pirfenidone (e.g., an extended-release pill or sustained-release pill (e.g., an extended-release tablet or sustained-release tablet of pirfenidone)) to the subject, wherein the subject has liver contraindication(s) (e.g., liver disease, liver damage, or a liver injury) for standard immediate-release pirfenidone and has a high viral burden (e.g., low levels of cycle threshold (Ct)) and elevated levels of one or more markers of inflammation (e.g., pro-inflammatory cytokines, C-reactive protein (CRP), ferritin, and lactic acid dehydrogenase (LDH)). The present disclosure provides methods of treating and/or preventing viremia-induced disease (e.g., caused by SARS-CoV-2). In certain embodiments, the viremia caused by a virus and/or the viremia-induced disease is caused by an RNA virus (e.g., coronavirus). In certain embodiments, the viremia caused by a virus is caused by a DNA virus. In certain embodiments, the viremia caused by a virus is caused by an RNA virus (e.g., coronavirus). In certain embodiments, the viremia caused by a virus and/or the viremia-induced disease is caused by a coronavirus (e.g., SARS-CoV-2, SARS-CoV, MERS-CoV).

In certain embodiments, the viremia caused by a virus and/or the viremia-induced disease is caused by a coronavirus (e.g., SARS-CoV-2 and/or variant thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations), SARS-CoV, MERS-CoV). In certain embodiments, the viremia caused by a virus is caused by a coronavirus (e.g., SARS-CoV-2, SARS-CoV, MERS-CoV). In certain embodiments, the viremia caused by a virus and/or the viremia-induced disease is caused by SARS-CoV-2, SARS-CoV, or MERS-CoV. In certain embodiments, the viremia caused by a virus is caused by SARS-CoV-2, SARS-CoV, or MERS-CoV. In certain embodiments, the viremia caused by a virus and/or the viremia-induced disease is caused by SARS-CoV-2. In certain embodiments, the viremia caused by a virus is caused by SARS-CoV-2. In certain embodiments, the coronavirus is SARS-CoV-2.

In certain embodiments, the coronavirus is SARS-CoV. In certain embodiments, the coronavirus is MERS-CoV. In certain embodiments, the coronavirus causes coronavirus disease 2019 (COVID-19). In certain embodiments, the coronavirus causes severe acute respiratory syndrome (SARS). In certain embodiments, the coronavirus causes Middle East respiratory syndrome (MERS). In certain embodiments, the viremia (e.g., virus caused by an RNA virus (e.g., a coronavirus, for example, SARS-CoV-2, SARS-CoV, or MERS-CoV) is selected from the group consisting of primary viremia, secondary viremia, active viremia, and passive viremia. In certain embodiments, the viremia is primary viremia. In certain embodiments, the viremia is secondary viremia. In certain embodiments, the viremia is active viremia. In certain embodiments, the viremia is passive viremia.

In another aspect, provided are methods of treating and/or preventing an infection with a virus in a subject in need thereof, the method comprising administering pirfenidone (e.g., a sustained-release pill (e.g., a sustained-release tablet of pirfenidone)) to the subject. In another aspect, provided are methods of treating an infection with a virus in a subject in need thereof, the method comprising administering pirfenidone (e.g., a sustained-release pill (e.g., a sustained-release tablet of pirfenidone)) to the subject. In certain embodiments, the infection with a virus is infection with a DNA virus. In certain embodiments, the infection with a virus is infection with an RNA virus (e.g., coronavirus). In certain embodiments, the infection with a virus is infection with a coronavirus (e.g., SARS-CoV-2, SARS-CoV, MERS-CoV). In certain embodiments, the infection with a virus is infection with SARS-CoV-2, SARS-CoV, or MERS-CoV. In certain embodiments, the infection with a virus is infection with SARS-CoV-2. In certain embodiments, the coronavirus is SARS-CoV-2. In certain embodiments, the infection with a virus is infection with SARS-CoV-2 and/or SARS-CoV-2 variant thereof (e.g., SARS-CoV-2 variants of B.1.1.7, B.1.351, B.1.525, B.1.526, P.1, B.1.427, B.1.429, and/or SARS-CoV-2 variants with the Q677P and/or L445R mutations), SARS-CoV, or MERS-CoV. In certain embodiments, the coronavirus is SARS-CoV. In certain embodiments, the coronavirus is MERS-CoV. In certain embodiments, the coronavirus causes coronavirus disease 2019 (COVID-19). In certain embodiments, the coronavirus causes severe acute respiratory syndrome (SARS). In certain embodiments, the coronavirus causes Middle East respiratory syndrome (MERS).

In another aspect, provided are methods of inhibiting an inflammatory response by one or more pro-inflammatory cytokines (e.g., TNF-α, IFN-γ, IL-1β, IL-6, IL-12) in a subject in need thereof, the method comprising administering pirfenidone (e.g., a sustained-release pill (e.g., a sustained-release tablet of pirfenidone)) to the subject. In certain embodiments, the method comprises blocking and/or reducing an inflammatory response (e.g., a storm and/or a cascade by one or more pro-inflammatory cytokines (e.g., TNF-α, IFN-γ, IL-1β, IL-6, IL-12)). In certain embodiments, the method comprises reducing elevated levels of one or more markers of inflammation (e.g., D-dimers, C-reactive protein (CRP), ferritin, lactic acid dehydrogenase (LDH)) in a subject in need thereof (e.g., with viremia caused by a virus), In certain embodiments, the method comprises reducing elevated levels of D-dimers in a subject in need thereof (e.g., with viremia caused by a virus), In certain embodiments, the method comprises reducing elevated levels of C-reactive protein (CRP) in a subject in need thereof (e.g., with viremia caused by a virus), In certain embodiments, the method comprises reducing elevated levels of ferritin. In certain embodiments, the method comprises reducing elevated levels of lactic acid dehydrogenase (LDH)) in a subject in need thereof (e.g., with viremia caused by a virus), In certain embodiments, the method comprises reducing and/or inhibiting the inflammatory response, for example, wherein the inflammatory response is caused or mediated by one or more pro-inflammatory cytokines (e.g., TNF-α, IFN-γ, IL-10, IL-6, IL-12). In certain embodiments, the method comprises inhibiting the inflammatory response by one or more pro-inflammatory cytokines (e.g., TNF-α, IFN-γ, IL-1β, IL-6, IL-12). In certain embodiments, the inflammatory response results in acute respiratory distress syndrome (ARDS), pulmonary edema, poor lung oxygen transfer, decompensation, a need for mechanical breathing assistance, and/or multi-organ failure. In certain embodiments, the inflammatory response results in acute respiratory distress syndrome (ARDS), for example, progressive ARDS. In certain embodiments, the inflammatory response is induced by an infection with a virus, for example, an RNA virus (e.g., coronavirus). In certain embodiments, the infection with a virus is an infection with a coronavirus (e.g., SARS-CoV-2, SARS-CoV, MERS-CoV). In certain embodiments, the infection with a virus is an infection with SARS-CoV-2, SARS-CoV, or MERS-CoV. In certain embodiments, the infection with a virus is an infection with SARS-CoV-2 and/or a SARS-CoV-2 variant thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations), SARS-CoV, or MERS-CoV. In certain embodiments, the infection with a virus is infection with SARS-CoV-2. In certain embodiments, the coronavirus is SARS-CoV-2. In certain embodiments, the coronavirus is SARS-CoV. In certain embodiments, the coronavirus is MERS-CoV. In certain embodiments, the coronavirus causes coronavirus disease 2019 (COVID-19). In certain embodiments, the coronavirus causes severe acute respiratory syndrome (SARS). In certain embodiments, the coronavirus causes Middle East respiratory syndrome (MERS).

In another aspect, provided are methods of reducing elevated levels of one or more markers of inflammation (e.g., D-dimers, C-reactive protein (CRP), ferritin, lactic acid dehydrogenase (LDH)) in a subject in need thereof (e.g., with viremia caused by a virus), wherein the subject has liver contraindication(s) (e.g., liver disease, liver damage, or a liver injury) for standard immediate-release pirfenidone and has a high viral burden (e.g., low levels of cycle threshold (Ct)) and elevated levels of one or more markers of inflammation (e.g., pro-inflammatory cytokines, C-reactive protein (CRP), ferritin, and lactic acid dehydrogenase (LDH)), In certain embodiments, provided are methods of reducing elevated levels of D-dimers in a subject in need thereof (e.g., with viremia caused by a virus), In certain embodiments, provided are methods of reducing C-reactive protein (CRP) in a subject in need thereof (e.g., with viremia caused by a virus), In certain embodiments, provided are methods of reducing ferritin in a subject in need thereof (e.g., with viremia caused by a virus), In certain embodiments, provided are methods of reducing lactic acid dehydrogenase (LDH)) in a subject in need thereof (e.g., with viremia caused by a virus), In another aspect, provided are methods of inhibiting a cascade by one or more pro-inflammatory cytokines (e.g., TNF-α, IFN-γ, IL-1β, IL-6, IL-12) in a subject in need thereof, the method comprising administering pirfenidone (e.g., a sustained-release pill (e.g., a sustained-release tablet of pirfenidone)) to the subject. In another aspect, provided are methods of inhibiting a cascade by one or more pro-inflammatory cytokines (e.g., TNF-α, IFN-γ, IL-1, IL-1, IL-2, IL-6, IL-12, IL-17, IL-18, chemokines, IL-7, IL-10, granulocyte-colony-stimulating factor, IP-10, monocyte chemoattractant protein-1 (MCP-1), macrophage inflammatory protein-1A (MIP-1A)).

### Pharmaceutical Compositions and Kits for Use

In another aspect, provided is pirfenidone, for example, in pharmaceutical compositions (e.g., immediate-release formulations or modified-release formulations, including, for example, extended-release formulations, sustained-release formulations, controlled-release formulations (e.g., an extended-release or sustained-release pill (e.g., an extended-release tablet or sustained-release tablet comprising approximately 100 mg to approximately 850 mg of pirfenidone) for use in treating and/or preventing COVID-19, viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), treating and/or preventing an infection with a virus (e.g., RNA virus, for example, coronavirus (e.g., severe acute respiratory syndrome coronavirus-2 (SARS-CoV-2) and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus). In certain embodiments, the ARDS is caused by infection with an infectious agent (e.g., a virus, for example, RNA virus). In certain embodiments, the ARDS is caused by infection with coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)). In certain embodiments, in the pirfenidone pharmaceutical composition, the pirfenidone is formulated in one or more sustained-release pills (e.g., sustained-release tablets), wherein each of the one or more sustained-release pills (e.g., sustained-release tablets) comprises the pirfenidone and the one or more excipients. In another aspect, provided are pirfenidone pharmaceutical compositions (e.g., sustained-release pirfenidone formulations, for example, a sustained-release pill (e.g., a sustained-release tablet comprising approximately 100 mg to approximately 850 mg of pirfenidone) for use in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus). In certain embodiments, the ARDS is caused by infection with an infectious agent (e.g., a virus, for example, RNA virus). In certain embodiments, the ARDS is caused by infection with coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)). In certain embodiments, in the pirfenidone pharmaceutical composition, the pirfenidone is formulated in one or more sustained-release pills (e.g., sustained-release tablets), wherein each of the one or more sustained-release pills (e.g., sustained-release tablets) comprises the pirfenidone and the one or more excipients. In certain embodiments, in the pirfenidone pharmaceutical composition, the pirfenidone is formulated as an immediate-release formulations or modified-release formulations, including, for example, extended-release formulations, sustained-release formulations, controlled-release formulations (e.g., an extended-release or sustained-release pill (e.g., an extended-release tablet or sustained-release tablet)) wherein each of the one or more extended-release pills (e.g., extended-release tablets) comprises the pirfenidone and the one or more excipients.

In certain embodiments, the pirfenidone pharmaceutical composition (e.g., immediate-release formulations or modified-release formulations, including, for example, extended-release formulations, sustained-release formulations, controlled-release formulations (e.g., an extended-release or sustained-release pill (e.g., an extended-release tablet or sustained-release tablet comprising approximately 100 mg to approximately 850 mg of pirfenidone) is used in treating and/or preventing viremia, for example, viremia caused by an RNA virus (e.g., coronavirus) or DNA virus. In certain embodiments, the pirfenidone pharmaceutical composition (e.g., sustained-release pirfenidone formulations, for example, a sustained-release pill (e.g., a sustained-release tablet comprising approximately 100 mg to approximately 850 mg of pirfenidone) is used in treating and/or preventing viremia, for example, viremia caused by an RNA virus (e.g., coronavirus) or DNA virus. In certain embodiments, the viremia caused by a virus and/or the viremia-induced disease (e.g., caused by SARS-CoV-2) is caused by a coronavirus (e.g., SARS-CoV-2, SARS-CoV, MERS-CoV).

In certain embodiments, the viremia caused by a virus and/or the viremia-induced disease (e.g., caused by SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)) is caused by a coronavirus (e.g., SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations), SARS-CoV, MERS-CoV). In certain embodiments, the viremia caused by a virus and/or the viremia-induced disease (e.g., caused by SARS-CoV-2) is caused by SARS-CoV-2, SARS-CoV, or MERS-CoV. In certain embodiments, the viremia caused by a virus and/or the viremia-induced disease (e.g., caused by SARS-CoV-2) is caused by SARS-CoV-2. In certain embodiments, the coronavirus is SARS-CoV-2. In certain embodiments, the coronavirus is SARS-CoV. In certain embodiments, the coronavirus is MERS-CoV. In certain embodiments, the coronavirus causes coronavirus disease 2019 (COVID-19). In certain embodiments, the coronavirus causes severe acute respiratory syndrome (SARS). In certain embodiments, the coronavirus causes Middle East respiratory syndrome (MERS). In certain embodiments, the viremia (e.g., virus caused by an RNA virus (e.g., coronavirus, for example, SARS-CoV-2, SARS-CoV, or MERS-CoV) is selected from the group consisting of primary viremia, secondary viremia, active viremia, and passive viremia. In certain embodiments, the viremia is primary viremia. In certain embodiments, the viremia is secondary viremia. In certain embodiments, the viremia is active viremia. In certain embodiments, the viremia is passive viremia. In certain embodiments, the pirfenidone pharmaceutical composition (e.g., sustained-release pirfenidone formulations, for example, a sustained-release pill (e.g., a sustained-release tablet comprising approximately 100 mg to approximately 850 mg of pirfenidone) is used in treating and/or preventing an infection with a virus in a subject in need thereof, the method comprising administering pirfenidone (e.g., a sustained-release pill (e.g., a sustained-release tablet of pirfenidone)) to the subject. In certain embodiments, the infection with a virus is infection with an RNA virus (e.g., coronavirus). In certain embodiments, the infection with a virus is infection with a coronavirus (e.g., SARS-CoV-2, SARS-CoV, MERS-CoV). In certain embodiments, the infection with a virus is infection with SARS-CoV-2, SARS-CoV, or MERS-CoV. In certain embodiments, the infection with a virus is infection with SARS-CoV-2.

In certain embodiments, the methods described herein comprise a formulation, for example, a modified release formulation (e.g., extended-release, sustained-release, controlled release formulation, e.g., extended-release tablet) which releases pirfenidone over approximately 8-9 hours, approximately 9-10 hours, or approximately 10-11 hours, approximately 10-11 hours, or approximately 10-12 hours, for example, over 12 hours. In certain embodiments, the methods described herein comprise a sustained release of pirfenidone over approximately 8-9 hours, approximately 9-10 hours, or approximately 10-11 hours, approximately 10-11 hours, or approximately 10-12 hours. In certain embodiments, the methods described herein comprise a sustained release of pirfenidone over approximately 12 hours. In certain embodiments, the modified release decrease side effects in patients with contraindications (e.g., liver contraindications, e.g., liver disease, liver damage, or a liver injury) for standard immediate-release pirfenidone.

In certain embodiments, the administered pirfenidone is formulated in a sustained-release pharmaceutical composition. In certain embodiments, the administered pirfenidone is formulated in a modified-release (e.g., extended-release, sustained-release) pharmaceutical composition. In certain embodiments, the administered pirfenidone is formulated in one or more sustained-release pills (e.g., sustained-release tablets), wherein each of the one or more sustained-release pills comprises pirfenidone and one or more excipients. In certain embodiments, the administered pirfenidone is formulated in one or more immediate-release formulations or modified-release formulations, including, for example, extended-release formulations, sustained-release formulations, controlled-release formulations (e.g., an extended-release or sustained-release pill, wherein each of the one or more modified-release pills (e.g., extended-release or sustained-release tablets comprises approximately 100 mg to approximately 1000 mg of pirfenidone, approximately 100 mg to approximately 200 mg of pirfenidone, approximately 200 mg to approximately 300 mg of pirfenidone, approximately 300 mg to approximately 400 mg of pirfenidone, approximately 400 mg to approximately 500 mg of pirfenidone, approximately 500 mg to approximately 600 mg of pirfenidone, approximately 200 mg to approximately 600 mg of pirfenidone, approximately 600 mg to approximately 700 mg of pirfenidone, approximately 700 mg to approximately 800 mg of pirfenidone, approximately 600 mg to approximately 850 mg of pirfenidone, approximately 700 mg to approximately 850 mg of pirfenidone, approximately 800 mg to approximately 900 mg of pirfenidone, approximately 800 mg to approximately 1000 mg of pirfenidone, approximately 900 mg to approximately 1000 mg of pirfenidone, approximately 1000 mg to approximately 1100 mg of pirfenidone, approximately 1100 mg to approximately 1200 mg of pirfenidone, approximately 1200 mg to approximately 1300 mg of pirfenidone, approximately 1300 mg to approximately 1400 mg of pirfenidone, approximately 1400 mg to approximately 1500 mg of pirfenidone, approximately 800 mg to approximately 1200 mg of pirfenidone, or approximately 800 mg to approximately 1500 mg of pirfenidone, or approximately 600 mg to approximately 1500 mg of pirfenidone. In certain embodiments, each of the one or more sustained-release pills (e.g., sustained-release tablets) comprises approximately 100 mg to approximately 1000 mg of pirfenidone, approximately 100 mg to approximately 200 mg of pirfenidone, approximately 200 mg to approximately 300 mg of pirfenidone, approximately 300 mg to approximately 400 mg of pirfenidone, approximately 400 mg to approximately 500 mg of pirfenidone, approximately 500 mg to approximately 600 mg of pirfenidone, approximately 200 mg to approximately 600 mg of pirfenidone, approximately 600 mg to approximately 700 mg of pirfenidone, approximately 700 mg to approximately 800 mg of pirfenidone, approximately 600 mg to approximately 850 mg of pirfenidone, approximately 700 mg to approximately 850 mg of pirfenidone, approximately 800 mg to approximately 900 mg of pirfenidone, approximately 800 mg to approximately 1000 mg of pirfenidone, approximately 900 mg to approximately 1000 mg of pirfenidone, approximately 1000 mg to approximately 1100 mg of pirfenidone, approximately 1100 mg to approximately 1200 mg of pirfenidone, or approximately 800 mg to approximately 1200 mg of pirfenidone. In certain embodiments, each of the one or more sustained-release pills (e.g., sustained-release tablets) comprises approximately 100 mg to approximately 850 mg of pirfenidone. In certain embodiments, each of the one or more sustained-release pills (e.g., sustained-release tablets) comprises approximately 400 mg of pirfenidone, approximately 500 mg of pirfenidone, approximately 600 mg of pirfenidone, approximately 700 mg of pirfenidone, approximately 500 mg of pirfenidone, approximately 800 mg of pirfenidone, approximately 850 mg of pirfenidone, approximately 900 mg of pirfenidone, approximately 1000 mg of pirfenidone, approximately 1200 mg of pirfenidone, approximately 1400 mg of pirfenidone, approximately 1600 mg of pirfenidone, or approximately 1800 mg of pirfenidone. In certain embodiments, the administered pirfenidone is formulated in an extended-release pill which comprises: a) approximately 100 mg to approximately 850 mg (e.g., approximately 600 mg) of pirfenidone; b) approximately 100 mg to 125 mg (e.g., approximately 118.8 mg) microcrystalline cellulose; c) approximately 50.0 mg to 100.0 mg (e.g., approximately 68.0 mg, approximately 70.0 mg, approximately 71.0 mg) low viscosity HPMC; d) approximately 30.0 mg to 50.0 mg (e.g., approximately 45 mg, approximately 46 mg, approximately 46.5 mg) high viscosity HPMC; e) approximately 5.0 mg to 10.0 mg (e.g., approximately 8.0 mg, approximately 8.25 mg, approximately 8.5 mg, approximately 9.0 mg) silicon dioxide; and f) approximately 5.0 mg to 10.0 mg (e.g., approximately 5.0 mg, approximately 5.25 mg, approximately 5.5 mg, approximately 6.0 mg, approximately 6.1 mg, approximately 6.2 mg, approximately 6.3 mg, approximately 6.4 mg) sodium stearyl fumarate. In certain embodiments, the administered pirfenidone is formulated in a sustained-release pill, for example, a sustained-release tablet, sustained-release capsule, or sustained-release gel capsule. In certain embodiments, the administered pirfenidone is formulated in a sustained-release pill, for example, a sustained-release tablet, capsule, or gel capsule. In certain embodiments, the administered pirfenidone is formulated in an extended-release tablet. In certain embodiments, the administered pirfenidone is formulated in an extended-release tablet of pirfenidone XR (Kitoscell® LP). In certain embodiments, the administered pirfenidone is formulated in a sustained-release tablet of Kitoscell® LP. The formulation of Kitoscell® LP is disclosed in U.S. patent application U.S. Ser. No. 14/233,600, filed May 20, 2014, issued as U.S. Pat. No. 9,408,836 on Aug. 9, 2016; U.S. patent application U.S. Ser. No. 15/177,760, filed Jun. 9, 2016, issued as U.S. Pat. No. 9,962,374 on May 8, 2018; U.S. patent application U.S. Ser. No. 15/831,650, filed Dec. 5, 2017, issued as U.S. Pat. No. 10,383,862 on Aug. 20, 2019; U.S. patent application U.S. Ser. No. 16/460,407, filed Jul. 2, 2019; and U.S. patent application U.S. Ser. No. 16/544,643, filed Aug. 19, 2019; each of which is incorporated herein by reference.

In certain embodiments, the subject being treated by the methods disclosed herein develops and/or has fibrosis (e.g., lung fibrosis, liver fibrosis). In certain embodiments, the subject being treated by the methods disclosed herein develops fibrosis (e.g., lung fibrosis, liver fibrosis). In certain embodiments, the subject being treated by the methods disclosed herein has one or more contraindications for standard-release (immediate release) pirfenidone. In certain embodiments, the subject being treated by the methods disclosed herein has at least a liver contraindication for standard-release pirfenidone. In certain embodiments, the subject being treated by the methods disclosed herein has liver disease, liver damage, and/or a liver injury. In certain embodiments, the subject has liver disease with a Pugh-Child score of class A, class B, or class C. In certain embodiments, the subject has chronic liver disease. In certain embodiments, the subject has severe liver disease. In certain embodiments, the subject being treated by the methods disclosed herein has COVID-2019 (COVID-19). In certain embodiments, the subject being treated by the methods disclosed herein has elevated levels of one or more markers of inflammation (e.g., pro-inflammatory cytokines, C-reactive protein (CRP), ferritin, and lactic acid dehydrogenase (LDH)). In certain embodiments, the subject has high viral burden (e.g., low levels of cycle threshold (Ct)) and elevated levels of one or more markers of inflammation (e.g., pro-inflammatory cytokines, C-reactive protein (CRP), ferritin, and lactic acid dehydrogenase (LDH)). In certain embodiments, the subject with high viral burden has low levels of cycle threshold (Ct). In certain embodiments, the subject has liver contraindication(s) (e.g., liver disease, liver damage, or a liver injury) for standard immediate-release pirfenidone and has a high viral burden (e.g., low levels of cycle threshold (Ct)) and elevated levels of one or more markers of inflammation (e.g., pro-inflammatory cytokines, C-reactive protein (CRP), ferritin, and lactic acid dehydrogenase (LDH)). In certain embodiments, the subject has elevated levels of one or more markers of inflammation selected from the group consisting of C-reactive protein (CRP), ferritin, and lactic acid dehydrogenase (LDH). In certain embodiments, the subject has elevated levels of the one or more markers of inflammation comprising C-reactive protein (CRP), ferritin, and lactic acid dehydrogenase (LDH). In certain embodiments, the subject has elevated levels of one or more pro-inflammatory cytokines (e.g., TNF-α, IFN-γ, IL-1β, IL-6, and/or IL-12). In certain embodiments, the subject has pneumonia. In certain embodiments, the subject has lung inflammation (e.g., severe lung inflammation) and/or lung fibrosis. In certain embodiments, the subject has ARDS. In certain embodiments, the subject is over age 18. In certain embodiments, the subject is over age 14, over age 15, over age 16, over age 17, or over age 18, or over age 19. In certain embodiments, the subject is over age 10, over age 12, over age 14, over age 15, over age 16, over age 17, or over age 18, or over age 19, over age 20, over age 25, over age 30, over age 35, over age 40, over age 45, over age 50, over age 55, over age 60, over age 65, over age 70, over age 75, over age 80, over age 85, or over age 90. In certain embodiments, the subject is over age 20, over age 30, over age 40, over age 50, over age 60, over age 70, over age 80, or over age 90. In certain embodiments, the subject is over age 60. In certain embodiments, the subject is over age 65. In certain embodiments, the subject is over age 70. In certain embodiments, the subject is over age 75. In certain embodiments, the subject has one or more contraindications for standard immediate-release pirfenidone (e.g., liver disease, liver damage, or a liver injury), is above approximately 12 years of age, above approximately 14 years of age, above approximately 16 years of age, or is between approximately 16 years and 85 years of age, above approximately 20 years of age, above approximately 30 years of age, above approximately 40 years of age, above approximately 50 years of age, above approximately 60 years of age, above approximately 70 years of age, has a moderate or severe stage of COVID-19, and has elevated levels of one or more markers of inflammation (e.g., C-reactive protein, ferritin, LDH), has high viral load (e.g., high load of SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations).

In certain embodiments, the pirfenidone described herein is formulated in one or more immediate-release or modified-release pills (e.g., extended-release tablets, sustained-release tablets), wherein each of the one or more extended-release pills (e.g., extended-release tablets) comprises the pirfenidone and one or more excipients; the coronavirus is severe acute respiratory syndrome coronavirus-2 (SARS-CoV-2) and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations); and the subject has or had coronavirus disease 2019 (COVID-19). In certain embodiments, the pirfenidone described herein is formulated in one or more sustained-release pills (e.g., sustained-release tablets), wherein each of the one or more sustained-release pills (e.g., sustained-release tablets) comprises the pirfenidone and one or more excipients; the coronavirus is severe acute respiratory syndrome coronavirus-2 (SARS-CoV-2); and the subject has coronavirus disease 2019 (COVID-19). In certain embodiments, the subject has one or more contraindications for standard-release pirfenidone.

Solid dosage forms of the pirfenidone formulation for oral administration include capsules, tablets, pills, powders, and granules. In such solid dosage forms, pirfenidone is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Pharmaceutically acceptable excipients used in the manufacture of provided pharmaceutical compositions and/or formulations described herein include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition and/or formulation.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In certain embodiments, the one or more excipients comprise one or more excipients selected from the group consisting of a compression binder, diluent, disintegrant, coating agent, rate-controlling polymer, anti-caking agent, adsorbent, lubricant, and glidant. In certain embodiments, at least one excipient of the one or more excipients is selected from the group consisting of hydroxypropylmethylcellulose (HPMC), microcrystalline cellulose, silicon dioxide, and sodium stearyl fumarate. In certain embodiments, each of the one or more sustained-release pills (e.g., sustained-release tablets) comprises: a) approximately 100 mg to approximately 850 mg of pirfenidone; b) microcrystalline cellulose; c) low viscosity HPMC; d) high viscosity HPMC; e) silicon dioxide; and f) sodium stearyl fumarate. In certain embodiments, each of the one or more sustained-release tablets comprises: a) approximately 100 mg to approximately 850 mg of pirfenidone; b) microcrystalline cellulose; c) low viscosity HPMC; d) high viscosity HPMC; e) silicon dioxide; and f) sodium stearyl fumarate. In certain embodiments, each of the one or more sustained-release pills (e.g., sustained-release tablets) comprises: a) approximately 100 mg to approximately 850 mg of pirfenidone; b) approximately 75 mg to approximately 200 mg of microcrystalline cellulose; c) approximately 50 mg to approximately 150 mg of low viscosity HPMC; d) approximately 20 mg to approximately 100 mg of high viscosity HPMC; e) approximately 1.0 mg to approximately 15.0 mg of silicon dioxide; and f) approximately 1.0 mg to approximately 15.0 mg of sodium stearyl fumarate. In certain embodiments, the administered pirfenidone is formulated in a sustained-release tablet of Kitoscell® LP. The formulation of Kitoscell® LP and its excipients is disclosed in U.S. patent application U.S. Ser. No. 14/233,600, filed May 20, 2014, issued as U.S. Pat. No. 9,408,836 on Aug. 9, 2016; U.S. patent application U.S. Ser. No. 15/177,760, filed Jun. 9, 2016, issued as U.S. Pat. No. 9,962,374 on May 8, 2018; U.S. patent application U.S. Ser. No. 15/831,650, filed Dec. 5, 2017, issued as U.S. Pat. No. 10,383,862 on Aug. 20, 2019; U.S. patent application U.S. Ser. No. 16/460,407, filed Jul. 2, 2019; and U.S. patent application U.S. Ser. No. 16/544,643, filed Aug. 19, 2019; each of which is incorporated herein by reference.

### Dosages and Formulations

In certain embodiments, the administered pirfenidone formulated in a sustained-release pill (e.g., sustained-release tablet) with one or more excipients is administered in a daily dosage comprising approximately 400 mg to approximately 4800 mg of pirfenidone per day. In certain embodiments, the administered pirfenidone formulated in immediate-release formulations or modified-release formulations, including, for example, extended-release formulations, sustained-release formulations, controlled-release formulations (e.g., an extended-release or sustained-release pill (e.g., an extended-release tablet or sustained-release tablet with one or more excipients is administered in a daily dosage comprising approximately 400 mg to approximately 4800 mg of pirfenidone per day, a daily dosage comprising approximately 400 mg to approximately 600 mg of pirfenidone per day, a daily dosage comprising approximately 600 mg to approximately 1200 mg of pirfenidone per day, a daily dosage comprising approximately 600 mg to approximately 800 mg of pirfenidone per day, a daily dosage comprising approximately 800 mg to approximately 1200 mg of pirfenidone per day, a daily dosage comprising approximately 800 mg to approximately 900 mg of pirfenidone per day, a daily dosage comprising approximately 900 mg to approximately 1000 mg of pirfenidone per day, a daily dosage comprising approximately 1000 mg to approximately 2000 mg of pirfenidone per day, a daily dosage comprising approximately 2000 mg to approximately 2100 mg of pirfenidone per day, a daily dosage comprising approximately 2100 mg to approximately 2200 mg of pirfenidone per day, a daily dosage comprising approximately 2200 mg to approximately 2300 mg of pirfenidone per day, a daily dosage comprising approximately 2300 mg to approximately 2400 mg of pirfenidone per day, a daily dosage comprising approximately 2400 mg to approximately 2500 mg of pirfenidone per day, a daily dosage comprising approximately 2600 mg to approximately 2800 mg of pirfenidone per day, a daily dosage comprising approximately 2800 mg to approximately 3000 mg of pirfenidone per day, a daily dosage comprising approximately 3000 mg to approximately 3200 mg of pirfenidone per day, a daily dosage comprising approximately 3200 mg to approximately 3400 mg of pirfenidone per day, a daily dosage comprising approximately 3400 mg to approximately 3600 mg of pirfenidone per day, a daily dosage comprising approximately 3600 mg to approximately 3800 mg of pirfenidone per day, a daily dosage comprising approximately 3800 mg to approximately 4000 mg of pirfenidone per day, a daily dosage comprising approximately 4000 mg to approximately 4200 mg of pirfenidone per day, a daily dosage comprising approximately 4200 mg to approximately 4300 mg of pirfenidone per day, a daily dosage comprising approximately 4300 mg to approximately 4500 mg of pirfenidone per day, a daily dosage comprising approximately 4500 mg to approximately 4600 mg of pirfenidone per day, a daily dosage comprising approximately 4600 mg to approximately 4800 mg of pirfenidone per day, or a daily dosage comprising approximately 1000 mg to approximately 4800 mg of pirfenidone per day. In certain embodiments, the administered pirfenidone is formulated in a modified release formulation, for example, an extended-release pill (extended-release tablet) is administered immediately upon infection with a coronavirus (e.g., severe acute respiratory syndrome coronavirus-2 (SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)), and/or is administered before the inflammatory response is initiated, and/or is administered once the subject is infected with a coronavirus (e.g., severe acute respiratory syndrome coronavirus-2 (SARS-CoV-2 and/or variants thereof (e.g., SARS-CoV-2 variants of B.1.1.7, B.1.351, B.1.525, B.1.526, P.1, B.1.427, B.1.429, and/or SARS-CoV-2 variants with the Q677P and/or L452R mutations)) and shows early symptoms of COVID-19 (e.g., fatigue, headache, sore throat, chills, fever, cough, shortness of breath, loss of sense of taste and/or smell, chest pain, nasal congestion, runny nose, nausea, vomiting, diarrhea), and administered in a daily dosage comprising approximately 400 mg to approximately 4800 mg of pirfenidone per day, a daily dosage comprising approximately 400 mg to approximately 600 mg of pirfenidone per day, a daily dosage comprising approximately 600 mg to approximately 1200 mg of pirfenidone per day, a daily dosage comprising approximately 600 mg to approximately 800 mg of pirfenidone per day, a daily dosage comprising approximately 800 mg to approximately 1200 mg of pirfenidone per day, a daily dosage comprising approximately 800 mg to approximately 900 mg of pirfenidone per day, a daily dosage comprising approximately 900 mg to approximately 1000 mg of pirfenidone per day, a daily dosage comprising approximately 1000 mg to approximately 2000 mg of pirfenidone per day, a daily dosage comprising approximately 2000 mg to approximately 2100 mg of pirfenidone per day, a daily dosage comprising approximately 2100 mg to approximately 2200 mg of pirfenidone per day, a daily dosage comprising approximately 2200 mg to approximately 2300 mg of pirfenidone per day, a daily dosage comprising approximately 2300 mg to approximately 2400 mg of pirfenidone per day, a daily dosage comprising approximately 2400 mg to approximately 2500 mg of pirfenidone per day, a daily dosage comprising approximately 2600 mg to approximately 2800 mg of pirfenidone per day, a daily dosage comprising approximately 2800 mg to approximately 3000 mg of pirfenidone per day, a daily dosage comprising approximately 3000 mg to approximately 3200 mg of pirfenidone per day, a daily dosage comprising approximately 3200 mg to approximately 3400 mg of pirfenidone per day, a daily dosage comprising approximately 3400 mg to approximately 3600 mg of pirfenidone per day, a daily dosage comprising approximately 3600 mg to approximately 3800 mg of pirfenidone per day, a daily dosage comprising approximately 3800 mg to approximately 4000 mg of pirfenidone per day, a daily dosage comprising approximately 4000 mg to approximately 4200 mg of pirfenidone per day, a daily dosage comprising approximately 4200 mg to approximately 4300 mg of pirfenidone per day, a daily dosage comprising approximately 4300 mg to approximately 4500 mg of pirfenidone per day, a daily dosage comprising approximately 4500 mg to approximately 4600 mg of pirfenidone per day, a daily dosage comprising approximately 4600 mg to approximately 4800 mg of pirfenidone per day, or a daily dosage comprising approximately 1000 mg to approximately 4800 mg of pirfenidone per day; which is administered for approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 24 days or more, approximately 25 days or more, approximately 27 days or more, approximately 28 days or more, approximately 30 days or more, approximately 32 days or more, approximately 35 days or more, approximately 37 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, or approximately 100 days or more.

In certain embodiments, the administered pirfenidone formulated in a sustained-release pill (e.g., sustained-release tablet) with one or more excipients is administered in a daily dosage comprising approximately 400 mg to approximately 4800 mg of pirfenidone per day, a daily dosage comprising approximately 400 mg to approximately 600 mg of pirfenidone per day, a daily dosage comprising approximately 600 mg to approximately 1200 mg of pirfenidone per day, a daily dosage comprising approximately 600 mg to approximately 800 mg of pirfenidone per day, a daily dosage comprising approximately 800 mg to approximately 1200 mg of pirfenidone per day, a daily dosage comprising approximately 800 mg to approximately 900 mg of pirfenidone per day, a daily dosage comprising approximately 900 mg to approximately 1000 mg of pirfenidone per day, a daily dosage comprising approximately 1000 mg to approximately 2000 mg of pirfenidone per day, a daily dosage comprising approximately 2000 mg to approximately 2100 mg of pirfenidone per day, a daily dosage comprising approximately 2100 mg to approximately 2200 mg of pirfenidone per day, a daily dosage comprising approximately 2200 mg to approximately 2300 mg of pirfenidone per day, a daily dosage comprising approximately 2300 mg to approximately 2400 mg of pirfenidone per day, a daily dosage comprising approximately 2400 mg to approximately 2500 mg of pirfenidone per day, or a daily dosage comprising approximately 2500 mg to approximately 2600 mg of pirfenidone per day. In certain embodiments, the daily dosage of the administered pirfenidone formulation described herein is administered in a daily dosage comprising approximately 800 mg to approximately 3000 mg of pirfenidone per day. In certain embodiments, the daily dosage of the administered pirfenidone formulation described herein is administered in a daily dosage comprising approximately 1000 mg to approximately 2400 mg of pirfenidone per day.

In certain embodiments, the one or more sustained-release pirfenidone pills (e.g., sustained-release tablets) are administered orally 1 to 2 times per day, 2 to 3 times per day, 3 to 4 times per day, 4 to 5 times per day, 5 to 6 times per day, 6 to 7 times per day, 7 to 8 times per day, 8 to 9 times per day, 9 to 10 times per day, 1 to 8 times per day, or 1 to 10 times per day. In certain embodiments, the one or more sustained-release pirfenidone pills (e.g., sustained-release tablets) are administered orally one time per day, two times per day, three times per day, four times per day, or five times per day. In certain embodiments, the one or more sustained-release pirfenidone pills (e.g., sustained-release tablets) are administered orally four times per day. In certain embodiments, the one or more sustained-release pirfenidone tablets are formulated into a slurry and administered orally.

In certain embodiments, the one or more sustained-release pirfenidone pills (e.g., sustained-release tablets) are administered for approximately ten days or more. In certain embodiments, the one or more extended-release pirfenidone pills (e.g., extended-release tablets) are administered for approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 24 days or more, approximately 25 days or more, approximately 27 days or more, approximately 28 days or more, approximately 30 days or more, approximately 32 days or more, approximately 35 days or more, approximately 37 days or more, approximately 40 days or more, approximately 45 days or more, approximately 50 days or more, approximately 55 days or more, approximately 60 days or more, approximately 65 days or more, approximately 70 days or more, approximately 75 days or more, approximately 80 days or more, approximately 85 days or more, approximately 90 days or more, approximately 95 days or more, or approximately 100 days or more. In certain embodiments, the one or more sustained-release pirfenidone pills (e.g., sustained-release tablets) are administered for approximately 5 days or more, approximately 6 days or more, approximately 7 days or more, approximately 8 days or more, approximately 9 days or more, approximately 10 days or more, approximately 11 days or more, approximately 12 days or more, approximately 13 days or more, approximately 14 days or more, approximately 15 days or more, approximately 16 days or more, approximately 17 days or more, approximately 18 days or more, approximately 19 days or more, approximately 20 days or more, approximately 25 days or more, approximately 30 days or more, approximately 35 days or more, approximately 40 days or more, approximately 50 days or more, approximately 60 days or more, approximately 70 days or more, approximately 80 days or more, approximately 90 days or more, or approximately 100 days or more. In certain embodiments, the one or more sustained-release pirfenidone pills (e.g., sustained-release tablets) are administered for approximately 30 days or more. In certain embodiments, the one or more extended-release pirfenidone pills (e.g., extended-release tablets, sustained-release tablets) are administered for approximately 10-25 days, approximately 10-12 days, approximately 12-14 days, approximately 14-15 days, approximately 15-20 days, approximately 20-30 days, approximately 20-25 days, approximately 25-30 days, approximately 30-35 days, approximately 35-40 days, approximately 40-45 days, approximately 45-50 days, approximately 50-55 days, approximately 55-60 days, approximately 55-60 days, approximately 60-65 days, approximately 65-70 days, approximately 70-75 days, approximately 75-80 days, approximately 80-85 days, approximately 85-90 days, approximately 90-95 days, or approximately 95-10 days. In certain embodiments, the one or more sustained-release pirfenidone pills (e.g., sustained-release tablets) are administered for approximately 10-20 days, approximately 20-30 days, approximately 30-40 days, approximately 40-50 days, approximately 50-60 days, approximately 60-70 days, approximately 70-80 days, approximately 80-90 days, or approximately 90-10 days. In certain embodiments, the one or more sustained-release pirfenidone pills (e.g., sustained-release tablets) are administered for approximately 10 to approximately 90 days.

### Pharmacokinetic Properties of Pirfenidone Formulations

In certain embodiments, the maximum or peak concentration (Cₘₐₓ) from a first dose of the administered pirfenidone formulated in an extended-release or sustained-release pill (e.g., extended-release or sustained-release tablet), observed after administration, is between approximately 1.0 μg/mL to 4.0 μg/mL. In certain embodiments, the maximum or peak concentration (Cₘₐₓ) from a first dose of the administered pirfenidone formulated in a sustained-release pill (e.g., sustained-release tablet), observed after administration, is between approximately 1.0 μg/mL to 10.0 μg/mL. In certain embodiments, the maximum or peak concentration (Cₘₐₓ) from a first dose of the administered pirfenidone formulated in a sustained-release pill (e.g., sustained-release tablet) is between approximately 1.0 μg/mL to 2.0 μg/mL, between approximately 1.5 μg/mL to 3.0 μg/mL, between approximately 2.0 g/mL to 3.0 μg/mL, between approximately 2.5 μg/mL to 3.0 μg/mL, between approximately 3.0 μg/mL to 4.0 μg/mL, or between approximately 2.0 μg/mL to 4.0 μg/mL, or between approximately 1.0 μg/mL to 10.0 μg/mL. In certain embodiments, the maximum or peak concentration (Cₘₐₓ) from a first dose of the administered pirfenidone formulated in a sustained-release pill (e.g., sustained-release tablet) is approximately 2.478±0.66 μg/mL. In certain embodiments, the maximum or peak concentration (Cₘₐₓ) from a first dose of the administered pirfenidone formulated in an extended-release pill (e.g., extended-release tablet), observed after administration, is between approximately 1.0 μg/mL to 10.0 μg/mL. In certain embodiments, the maximum or peak concentration (Cₘₐₓ) from a first dose of the administered pirfenidone formulated in an extended-release pill (e.g., extended-release tablet) is between approximately 1.0 μg/mL to 2.0 μg/mL, between approximately 1.5 μg/mL to 3.0 μg/mL, between approximately 2.0 μg/mL to 3.0 μg/mL, between approximately 2.5 g/mL to 3.0 μg/mL, between approximately 3.0 μg/mL to 4.0 μg/mL, or between approximately 2.0 μg/mL to 4.0 μg/mL, or between approximately 1.0 μg/mL to 10.0 μg/mL. In certain embodiments, the maximum or peak concentration (Cₘₐₓ) from a first dose of the administered pirfenidone formulated in an extended-release pill (e.g., extended-release tablet) is approximately 2.478±0.66 μg/mL.

In certain embodiments, a Tₘₐₓ of the administered pirfenidone formulated in a sustained-release pill (e.g., sustained-release tablet) is between approximately 0.5 hours to 8.0 hours. In certain embodiments, a Tₘₐₓ of the administered pirfenidone formulated in a sustained-release pill (e.g., sustained-release tablet) is between approximately 1.5 hours to 8.0 hours. In certain embodiments, a Tₘₐₓ of the administered pirfenidone formulated in a sustained-release pill (e.g., sustained-release tablet) is between approximately 3.0 hours to approximately 3.5 hours, between approximately 3.0 hours to approximately 4.0 hours, between approximately 4.0 hours to approximately 5.0 hours or between approximately 3.0 hours to approximately 5.0 hours. In certain embodiments, a Tₘₐₓ of the administered pirfenidone formulated in a sustained-release pill (e.g., sustained-release tablet) is approximately 3.500±1.28 hours. In certain embodiments, a Tₘₐₓ of the administered pirfenidone formulated in an extended-release pill (e.g., extended-release tablet) is between approximately 0.5 hours to 8.0 hours. In certain embodiments, a Tₘₐₓ of the administered pirfenidone formulated in an extended-release pill (e.g., extended-release tablet) is between approximately 1.5 hours to 8.0 hours. In certain embodiments, a Tₘₐₓ of the administered pirfenidone formulated in an extended-release pill (e.g., extended-release tablet) is between approximately 3.0 hours to approximately 3.5 hours, between approximately 3.0 hours to approximately 4.0 hours, between approximately 4.0 hours to approximately 5.0 hours or between approximately 3.0 hours to approximately 5.0 hours. In certain embodiments, a Tₘₐₓ of the administered pirfenidone formulated in an extended-release pill (e.g., extended-release tablet) is approximately 3.500±1.28 hours.

In certain embodiments, the AUC₀₋ₜ of the administered pirfenidone formulated in a sustained-release pill (e.g., sustained-release tablet) is between approximately 10.0 g*hours/mL to approximately 25.0 μg*hours/mL or between approximately 10.0 g*hours/mL to approximately 30.0 μg*hours/mL. In certain embodiments, the AUC₀₋ₜ of the administered pirfenidone formulated in a sustained-release pill (e.g., sustained-release tablet) is between approximately 10.0 μg*hours/mL to approximately 30.0 μg*hours/mL. In certain embodiments, the AUC₀₋ₜ of the administered pirfenidone formulated in a sustained-release pill (e.g., sustained-release tablet) is approximately 21.213±8.07 μg*hours/mL. In certain embodiments, the AUC₀₋ₜ of the administered pirfenidone formulated in an extended-release pill (e.g., extended-release tablet) is between approximately 10.0 μg*hours/mL to approximately 25.0 μg*hours/mL or between approximately 10.0 μg*hours/mL to approximately 30.0 μg*hours/mL. In certain embodiments, the AUC₀₋ₜ of the administered pirfenidone formulated in an extended-release pill (e.g., extended-release tablet) is between approximately 10.0 μg*hours/mL to approximately 30.0 μg*hours/mL. In certain embodiments, the AUC₀₋ₜ of the administered pirfenidone formulated in an extended-release pill (e.g., extended-release tablet) is approximately 21.213±8.07 μg*hours/mL.

In certain embodiments, the half-life (T_{1/2}) of the administered pirfenidone formulated in a sustained-release pill (e.g., sustained-release tablet) is between approximately 1.6 hours to approximately 8.0 hours. In certain embodiments, the half-life (T_{1/2}) of the administered pirfenidone formulated in a sustained-release pill (e.g., sustained-release tablet) is between approximately 2.0 hours to approximately 8.0 hours. In certain embodiments, the half-life (T_{1/2}) of the administered pirfenidone formulated in a sustained-release pill (e.g., sustained-release tablet) is approximately 4.832±3.2 hours. In certain embodiments, the half-life (T_{1/2}) of the administered pirfenidone formulated in an extended-release pill (e.g., extended-release tablet) is between approximately 1.6 hours to approximately 8.0 hours. In certain embodiments, the half-life (T_{1/2}) of the administered pirfenidone formulated in an extended-release pill (e.g., extended-release tablet) is between approximately 2.0 hours to approximately 8.0 hours. In certain embodiments, the half-life (T_{1/2}) of the administered pirfenidone formulated in an extended-release pill (e.g., extended-release tablet) is approximately 4.832±3.2 hours.

In another aspect, provided are kits described herein that include a first container comprising pirfenidone pharmaceutical composition described herein. In certain embodiments, a kit described herein further includes instructions for using the pirfenidone pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating COVID-19 (e.g., in subjects with contraindications for immediate release pirfenidone, for example, with liver disease, liver damage, liver injury), and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus); inhibiting an inflammatory response by one or more pro-inflammatory cytokines (e.g., TNF-α, IFN-γ, IL-1β, IL-6, IL-12), reducing elevated levels of one or more markers of inflammation (e.g., C-reactive protein, ferritin, LDH) in a subject in need thereof with viremia caused by a virus, and/or inhibiting a cascade by one or more pro-inflammatory cytokines (e.g., TNF-α, IFN-γ, IL-1β, IL-6, IL-12), all in a subject in need thereof (e.g., a subject currently or previously infected with viremia caused by a virus, or currently having or previously had COVID-19).

In certain embodiments, provided are kits comprising pirfenidone pharmaceutical compositions (e.g., sustained-release pirfenidone formulations, for example, a sustained-release pill (e.g., a sustained-release tablet comprising approximately 100 mg to approximately 850 mg of pirfenidone and one or more excipients), for use in treating COVID-19, for use in treating and/or preventing viremia caused by a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), treating and/or preventing viremia-induced disease (e.g., caused by SARS-CoV-2), treating and/or preventing an infection with a virus (e.g., RNA virus, for example, coronavirus (e.g., severe acute respiratory syndrome coronavirus-2 (SARS-CoV-2), severe acute respiratory syndrome-associated coronavirus (SARS-CoV), Middle East respiratory syndrome coronavirus (MERS-CoV)), and/or treating and/or preventing ARDS caused by infection with an infectious agent (e.g., a virus, for example, RNA virus). In certain embodiments, provided are kits comprising: a pharmaceutical composition comprising pirfenidone, and one or more excipients, for use in treating or preventing viremia caused by a virus, viremia-induced disease, an infection with a virus, and/or ARDS caused by infection with an infectious agent; and instructions for administering to a subject or contacting a biological sample with the pharmaceutical composition.

### EXEMPLARY EMBODIMENTS

Embodiment 1. A method of treating or preventing viremia caused by a virus in a subject in need thereof, the method comprising administering pirfenidone to the subject.
Embodiment 2. A method of treating or preventing viremia-induced disease in a subject in need thereof, the method comprising administering pirfenidone to the subject.
Embodiment 3. A method of treating COVID-19 and/or COVID-19 disease caused by a SARS-CoV-2 variant thereof in a subject in need thereof, the method comprising administering pirfenidone to the subject.
Embodiment 4. A method of treating or preventing an infection with a virus in a subject in need thereof, the method comprising administering pirfenidone to the subject.
Embodiment 5. A method of inhibiting an inflammatory response by one or more pro-inflammatory cytokines in a subject in need thereof, the method comprising administering pirfenidone to the subject.
Embodiment 6. A method of inhibiting a cascade by one or more pro-inflammatory cytokines in a subject in need thereof, the method comprising administering pirfenidone to the subject.
Embodiment 7. A method of reducing elevated levels of one or more markers of inflammation in a subject in need thereof with viremia caused by a virus, the method comprising administering pirfenidone to the subject.
Embodiment 8. The method of any one of embodiments 1-7, wherein the pirfenidone is formulated in one or more extended-release pills, wherein each of the one or more extended-release pills comprises the pirfenidone and one or more excipients.
Embodiment 9. The method of any one of embodiments 1-8, wherein each of the one or more extended-release pills comprises approximately 100 mg to approximately 850 mg of pirfenidone.
Embodiment 10. The method of any one of embodiments 1-9, wherein each of the one or more extended-release pills comprises approximately 600 mg of pirfenidone.
Embodiment 11. The method of any one of embodiments 1-10, wherein a Cₘₐₓ from a first dose of the one or more extended-release pills is between approximately 1.0 μg/mL to 10.0 μg/mL.
Embodiment 12. The method of any one of embodiments 1-11, wherein the Cₘₐₓ from a first dose of the one or more extended-release pills is between approximately 1.0 μg/mL to 4.0 μg/mL.
Embodiment 13. The method of any one of embodiments 1-7, wherein the pirfenidone is formulated in one or more sustained-release pills, wherein each of the one or more sustained-release pills comprises the pirfenidone and one or more excipients.
Embodiment 14. The method of any one of embodiments 1-8 or 13, wherein each of the one or more sustained-release pills comprises approximately 100 mg to approximately 850 mg of pirfenidone.
Embodiment 15. The method of any one of embodiments 1-9 or 14, wherein each of the one or more sustained-release pills comprises approximately 600 mg of pirfenidone.
Embodiment 16. The method of any one of embodiments 1-10 or 15, wherein a Cₘₐₓ from a first dose of the one or more sustained-release pills is between approximately 1.0 g/mL to 10.0 μg/mL.
Embodiment 17. The method of any one of embodiments 1-11 or 16, wherein the Cₘₐₓ from a first dose of the one or more sustained-release pills is between approximately 1.0 g/mL to 4.0 μg/mL.
Embodiment 18. The method of any one of embodiments 1-17, wherein a Tₘₐₓ is between approximately 1.5 hours to 8.0 hours.
Embodiment 19. The method of any one of embodiments 1-18, wherein the Tₘₐₓ is between approximately 3.0 hours to approximately 5.0 hours.
Embodiment 20. The method of any one of embodiments 1-19, wherein an AUC₀₋ₜ is between approximately 10.0 μg*hours/mL to approximately 30.0 μg*hours/mL.
Embodiment 21. The method of any one of embodiments 1-20, wherein the half-life (T_{1/2}) is between approximately 1.6 hours to approximately 8.0 hours.
Embodiment 22. The method of any one of embodiments 7-21, wherein the one or more excipients comprise one or more excipients selected from the group consisting of a compression binder, diluent, disintegrant, coating agent, rate-controlling polymer, anti-caking agent, adsorbent, lubricant, and glidant.
Embodiment 23. The method of any one of embodiments 7-22, wherein at least one excipient of the one or more excipients is selected from the group consisting of hydroxypropylmethylcellulose (HPMC), microcrystalline cellulose, silicon dioxide, and sodium stearyl fumarate.
Embodiment 24. The method of any one of embodiments 7-23, wherein each of the one or more sustained-release pills comprises:
a) approximately 100 mg to approximately 850 mg of pirfenidone;
b) microcrystalline cellulose;
c) low viscosity HPMC;
d) high viscosity HPMC;
e) silicon dioxide; and
f) sodium stearyl fumarate.
Embodiment 25. The method of any one of embodiments 7-23, wherein each of the one or more extended-release pills comprises:
a) approximately 100 mg to approximately 850 mg of pirfenidone;
b) microcrystalline cellulose;
c) low viscosity HPMC;
d) high viscosity HPMC;
e) silicon dioxide; and
f) sodium stearyl fumarate.
Embodiment 26. The method of any one of embodiments 7-23 or 25, wherein each of the one or more extended-release pills comprises:
a) approximately 100 mg to approximately 850 mg of pirfenidone;
b) approximately 100 mg to 125 mg microcrystalline cellulose;
c) approximately 50.0 mg to 100.0 mg low viscosity HPMC;
d) approximately 30.0 mg to 50.0 mg high viscosity HPMC;
e) approximately 5.0 mg to 10.0 mg silicon dioxide; and
f) approximately 5.0 mg to 10.0 mg sodium stearyl fumarate.
Embodiment 27. The method of any one of embodiments 1-24, wherein the one or more sustained-release pills are formulated as tablets, capsules, or gel capsules.
28. The method of any one of embodiments 1-23 or 25-27, wherein the one or more extended-release pills are formulated as tablets, capsules, or gel capsules.
Embodiment 29. The method of any one of embodiments 1-23 or 25-28, wherein the one or more extended-release pills are formulated as tablets.
Embodiment 30. The method of any one of embodiments 1, 2, or 7-29, wherein the viremia is selected from the group consisting of primary viremia, secondary viremia, active viremia, and passive viremia.
Embodiment 31. The method of any one of embodiments 1, 2, or 7-30, wherein the viremia caused by the virus is viremia caused by an RNA virus or a DNA virus.
Embodiment 32. The method of any one of embodiments 2 or 7-31, wherein the viremia-induced disease is caused by an infection with a virus.
Embodiment 33. The method of any one of embodiments 5 or 8-32, wherein the inflammatory response is induced by an infection with a virus.
Embodiment 34. The method of any one of embodiments 4 or 8-33, wherein the infection with the virus is infection with an RNA virus or a DNA virus.
Embodiment 35. The method of embodiment 34, wherein the viremia caused by an RNA virus is viremia caused by a coronavirus.
Embodiment 36. The method of embodiment 34 or 35, wherein the infection with the RNA virus is an infection with a coronavirus.
Embodiment 37. The method of any one of embodiments 34-36, wherein the coronavirus is severe acute respiratory syndrome coronavirus-2 (SARS-CoV-2).
Embodiment 38. The method of any one of embodiments 34-36, wherein the coronavirus is a SARS-CoV-2 variant of B.1.1.7, B.1.351, B.1.525, B.1.526, P.1, B.1.427, B.1.429, and/or SARS-CoV-2 variants with the Q677P and/or L452R mutations.
Embodiment 39. The method of any one of embodiments 34-38, wherein the coronavirus causes coronavirus disease 2019 (COVID-19).
Embodiment 40. The method of any one of embodiments 34-38, wherein the coronavirus is severe acute respiratory syndrome-associated coronavirus (SARS-CoV).
Embodiment 41. The method of any one of embodiments 34-38, wherein the coronavirus is Middle East respiratory syndrome coronavirus (MERS-CoV).
Embodiment 42. The method of any one of embodiments 1-41, wherein the subject develops fibrosis.
Embodiment 43. The method of any one of embodiments 1-42, wherein the fibrosis is lung fibrosis or liver fibrosis.
Embodiment 44. The method of any one of embodiments 1-43, wherein the subject has one or more contraindications for standard immediate-release pirfenidone.
Embodiment 45. The method of any one of embodiments 1-44, wherein the subject has liver disease, liver damage, or a liver injury.
Embodiment 46. The method of any one of embodiments 1-45, wherein the subject has liver disease with a Pugh-Child score of class A, class B, or class C.
Embodiment 47. The method of any one of embodiments 45 or 46, wherein the liver disease is chronic liver disease.
Embodiment 48. The method of any one of embodiments 1-47, wherein the pirfenidone is formulated in one or more extended-release tablets, the subject has one or more liver contraindications for standard-release pirfenidone; and each of the one or more extended-release tablets comprises:
a) approximately 100 mg to approximately 850 mg of pirfenidone;
b) approximately 100 mg to 125 mg microcrystalline cellulose;
c) approximately 50.0 mg to 100.0 mg low viscosity HPMC;
d) approximately 30.0 mg to 50.0 mg high viscosity HPMC;
e) approximately 5.0 mg to 10.0 mg silicon dioxide; and
f) approximately 5.0 mg to 10.0 mg sodium stearyl fumarate.
Embodiment 49. The method of any one of embodiments 1-48, comprising blocking and/or reducing an inflammatory response.
Embodiment 50. The method of any one of embodiments 1-49, comprising inhibiting the inflammatory response by one or more pro-inflammatory cytokines.
Embodiment 51. The method of any one of embodiments 1-50, wherein the inflammatory response is a cascade by the one or more pro-inflammatory cytokines.
Embodiment 52. The method of any one of embodiments 1-51, wherein the inflammatory response results in acute respiratory distress syndrome (ARDS).
Embodiment 53. The method of any one of embodiments 1-52, wherein the subject has elevated levels of one or more markers of inflammation.
Embodiment 54. The method of embodiment 53, wherein the one or more markers of inflammation are selected from the group consisting of D-dimers, C-reactive protein (CRP), ferritin, and lactic acid dehydrogenase (LDH).
Embodiment 55. The method of any one of embodiments 1-54, wherein the one or more markers of inflammation comprise C-reactive protein (CRP), ferritin, and lactic acid dehydrogenase (LDH).
Embodiment 56. The method of any one of embodiments 1-55, wherein the subject has elevated levels of one or more pro-inflammatory cytokines.
Embodiment 57. The method of embodiment 56, wherein the one or more pro-inflammatory cytokines are selected from the group consisting of TNF-α, IFN-γ, IL-1β, IL-6, and/or IL-12.
Embodiment 58. The method of any one of embodiments 1-57, wherein the subject has high viral burden and elevated levels of one or more markers of inflammation.
Embodiment 59. The method of any one of embodiments 1-58, wherein the subject that has high viral burden has low levels of cycle threshold (Ct).
Embodiment 60. The method of any one of embodiments 1-59, wherein the subject has pneumonia.
Embodiment 61. The method of any one of embodiments 1-60, wherein the subject has lung inflammation and/or lung fibrosis.
Embodiment 62. The method of any one of embodiments 1-61, wherein the subject has acute respiratory distress syndrome (ARDS).
Embodiment 63. The method of any one of embodiments 1-62, wherein the subject has COVID-19.
Embodiment 64. The method of any one of embodiments 1-63, wherein the pirfenidone is formulated in one or more extended-release pills, wherein each of the one or more extended-release pills comprises the pirfenidone and one or more excipients; the coronavirus is severe acute respiratory syndrome coronavirus-2 (SARS-CoV-2) and/or a SARS-CoV-2 variant thereof; and the subject has coronavirus disease 2019 (COVID-19) and/or a disease caused by a SARS-CoV-2 variant thereof.
Embodiment 65. The method of any one of embodiments 1-63, wherein the pirfenidone is formulated in one or more sustained-release pills, wherein each of the one or more sustained-release pills comprises the pirfenidone and one or more excipients; the coronavirus is severe acute respiratory syndrome coronavirus-2 (SARS-CoV-2); and the subject has coronavirus disease 2019 (COVID-19).
Embodiment 66. The method of embodiment 64, wherein the subject has one or more contraindications for immediate-release pirfenidone.
Embodiment 67. The method of embodiment 64, wherein the subject has one or more contraindications for standard-release pirfenidone.
Embodiment 68. The method of any one of embodiments 1-67, wherein a daily dosage comprises approximately 400 mg to approximately 4800 mg of pirfenidone per day.
Embodiment 69. The method of any one of embodiments 1-68, wherein a daily dosage comprises approximately 800 mg to approximately 3000 mg of pirfenidone per day.
Embodiment 70. The method of any one of embodiments 1-69, wherein a daily dosage comprises approximately 1000 mg to approximately 2400 mg of pirfenidone per day.
Embodiment 71. The method of any one of embodiments 1-70, wherein the one or more sustained-release tablets are administered orally 1 to 8 times per day.
Embodiment 72. The method of any one of embodiments 1-71, wherein the one or more sustained-release pills are administered orally two times per day.
Embodiment 73. The method of any one of embodiments 1-72, wherein the one or more sustained-release pills are administered for approximately ten days or more.
Embodiment 74. The method of any one of embodiments 1-70, wherein the one or more extended-release tablets are administered orally 1 to 8 times per day.
Embodiment 75. The method of any one of embodiments 1-70 or 74, wherein the one or more extended-release pills are administered orally two times per day.
Embodiment 76. The method of any one of embodiments 1-70 or 75, wherein the one or more extended-release pills are administered for approximately ten days or more.
Embodiment 77. The method of any one of embodiments 1-70 or 74-76, wherein the one or more extended-release pills are administered for approximately fourteen days or more. Embodiment 78. The method of any one of embodiments 1-70 or 74-76, wherein the one or more extended-release pills are administered for approximately twenty-eight days or more.
Embodiment 79. The method of any one of embodiments 1-73, wherein the one or more sustained-release pills are administered for approximately thirty days or more.
Embodiment 80. The method of any one of embodiments 1-73 or 79, wherein the one or more sustained-release pills are administered for approximately ten to approximately thirty days.
Embodiment 81. The method of any one of embodiments 1-70 or 74-78, wherein the one or more extended-release pills are administered for approximately thirty days or more.
Embodiment 82. The method of any one of embodiments 1-70 or 74-78, wherein the one or more extended-release pills are administered for approximately ten to approximately thirty days.
Embodiment 83. The method of any one of embodiments 1-70, 74-78, 81, or 82, comprising a formulation of pirfenidone that provides release of pirfenidone over 12 hours.
Embodiment 84. A pharmaceutical composition comprising pirfenidone and one or more excipients, for use in treating or preventing viremia caused by a virus, viremia-induced disease, COVID-19, a disease caused by a SARS-CoV-2 variant thereof, an infection with a virus, and/or ARDS caused by infection with an infectious agent in a subject in need thereof.
Embodiment 85. The pharmaceutical composition of embodiment 84, comprising pirfenidone and one or more excipients, for use in treating or preventing viremia caused by a virus, viremia-induced disease, an infection with a virus, and/or ARDS caused by infection with an infectious agent in a subject in need thereof.
Embodiment 86. The pharmaceutical composition of embodiment 84 or 85, wherein the pirfenidone is formulated in one or more sustained-release pills, wherein each of the one or more sustained-release pills comprises the pirfenidone and the one or more excipients.
Embodiment 87. The pharmaceutical composition of embodiment 84 or 85, wherein the pirfenidone is formulated in one or more extended-release pills, wherein each of the one or more extended-release pills comprises the pirfenidone and the one or more excipients.
Embodiment 88. The pharmaceutical composition of embodiment 84-87, wherein the viremia-induced disease is caused by severe acute respiratory syndrome coronavirus-2 (SARS-CoV-2).
Embodiment 89. The pharmaceutical composition of embodiment 84-88, wherein the infection with the virus is infection with SARS-CoV-2.
Embodiment 90. The pharmaceutical composition of any one of embodiments 84-89, wherein the subject has COVID-19.
Embodiment 91. Use of pirfenidone for treating or preventing viremia caused by a virus, viremia-induced disease, COVID-19, a disease caused by a SARS-CoV-2 variant thereof, an infection with a virus, and/or ARDS caused by infection with an infectious agent in a subject in need thereof.
Embodiment 92. A kit comprising a pharmaceutical composition comprising pirfenidone and one or more excipients, for use in treating or preventing viremia caused by a virus, viremia-induced disease, COVID-19, a disease caused by a SARS-CoV-2 variant thereof, an infection with a virus, and/or ARDS caused by infection with an infectious agent; and

instructions for administering to a subject or contacting a biological sample with the pharmaceutical composition.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope. The formulations and compositions provided herein can be prepared from readily available starting materials using methods known in the art. For example, the formulations and compositions disclosed herein are prepared using methods for preparing tablets of Kitoscell® LP as disclosed in U.S. patent application U.S. Ser. No. 14/233,600, filed May 20, 2014, issued as U.S. Pat. No. 9,408,836 on Aug. 9, 2016; U.S. patent application U.S. Ser. No. 15/177,760, filed Jun. 9, 2016, issued as U.S. Pat. No. 9,962,374 on May 8, 2018; U.S. patent application U.S. Ser. No. 15/831,650, filed Dec. 5, 2017, issued as U.S. Pat. No. 10,383,862 on Aug. 20, 2019; U.S. patent application U.S. Ser. No. 16/460,407, filed Jul. 2, 2019; and U.S. patent application U.S. Ser. No. 16/544,643, filed Aug. 19, 2019; each of which is incorporated herein by reference.

### Example 1. KITOSCELL® LP as a Protector for COVID-19 Patients

The clinical deterioration in COVID-19 is in part due to the inflammatory response caused by the virus infection. This leads to pulmonary edema, poor lung oxygen transfer, decompensation, requirement for mechanical breathing assistance and eventually death for many patients. This in turn sequentially requires patient hospitalization, transfer to intensive care units (ICU) and frequent intubation. If the cytokine storm can be prophylactically avoided or minimized, the cascade of ARDS events and multi-organ failure may be averted.

Preclinical and clinical pharmacokinetic studies show that oral pirfenidone is very rapidly absorbed and metabolized. After oral administration in healthy adults, in standard immediate formulations, pirfenidone reaches its maximum levels in blood after 0.5 to 2 hours, if taken in fasting or post food, respectively, with a terminal half-life of approximately 2.5 hours (Rubino et al., 2009). This lead to high Cₘₐₓ levels, which may result in less tolerance and poor compliance, and low Cₘᵢₙ, which may cause periodic increases in cytokine levels and inflammation. KITOSCELL® LP provided decreased Cₘₐₓ levels, increased T_{1/2} and Cₘᵢₙ and equivalent AUC (see Table 1). The decreased Cₘₐₓ may avoid potential adverse events. The superior pharmacokinetic properties of KITOSCELL® LP, for example, increased T_{1/2} and higher levels at the trough (Cₘᵢₙ), may maintain more constant therapeutic levels of pirfenidone and therefore avoid the intermittent pro-inflammatory cytokine cascades that can lead to progressive ARDS.

TABLE 1
KITOSCELL ® LP (Pirfenidone XR) Slow Release Pharmacokinetics

| | Cₘₐₓ | Tₘₐₓ | AUC₀₋ₜ | T_{1/2} |
|---|---|---|---|---|
| Dose (mg) | (μg/mL) | (hr) | (μg · h/mL) | (hr) |
| 600 mg pirfenidone | 2.478 ± 0.66 | 3.500 ± 1.28 | 21.213 ± 8.07 | 4.832 ± 3.2 |
| in KITOSCELL ® LP | | | | |
| 600 standard formula | 10.57 ± 1.78 | 0.83 ± 0.26 | 37.03 ± 11.97 | 1.76 ± 0.40 |

Pharmacokinetic analysis of KITOSCELL® LP suggest a sustained-release pirfenidone formulation (e.g., KITOSCELL® LP) may improve the potential benefits of pirfenidone as a prophylactic and/or therapeutic agent in COVID-19 patients, including improving the anti-fibrotic and potent anti-inflammatory effects via cytokine regulation, with few side effects.

### Objectives

The objectives for the presently disclosed study are to evaluate safety and improvement in outcome for patients with COVID-19. The primary endpoint is a clinical severity assessment. The secondary endpoints include: safety based on standard laboratory and clinical adverse event monitoring; change in inflammatory markers such as serum LDH, ferritin, CRP, IL-6; change in SARS-CoV-2 viral load; and exploratory Immune biomarkers.

### Subject Population

### Inclusion Criteria

To evaluate KITOSCELL® LP in the diagnosed, hospitalized patient population, patients meeting the following criteria are considered for study inclusion: hospitalized patients with laboratory-confirmed SARS-CoV-2 infection as determined by polymerase chain reaction (PCR) or other commercial or public health assay or, hospitalized patients presumed positive (pending test result) for SARS-CoV-2 infection based on clinical presentation and recent exposure with SARS-CoV-2. Dosing may be started prior to test-confirmed SARS-CoV2 infection, once patient is consented.

To evaluate KITOSCELL® LP in the diagnosed, non-hospitalized patient population, patients meeting the following criteria are considered for study inclusion: non-hospitalized patients with laboratory-confirmed SARS-CoV-2 infection as determined by polymerase chain reaction (PCR) or other commercial or public health assay or, non-hospitalized patients presumed positive (pending test result) for SARS-CoV-2 infection based on clinical presentation and recent exposure with SARS-CoV-2. Dosing may be started prior to test-confirmed SARS-CoV2 infection, once patient is consented.

In addition to the above, patients must be: 18 years of age or older, able to swallow pills and willing to comply with study assessments, able to provide informed consent signed by patient or legally authorized representative, have total bilirubin ≤1.5× upper limit of normal (except for patients with known Gilbert disease who must have total bilirubin ≤3× upper limit of normal), and have ALT ≤5× upper limit of normal.

Exclusion Criteria

Patients are excluded from the study if they meet any of the following exclusion criteria: are intubated prior to consent; are planning treatment with anti-IL-6 or anti-IL-6R antagonists or Janus kinase inhibitors (JAKi) in the next 7 days; are currently undergoing treatment with disease-modifying anti-rheumatic drugs (DMARDs) or other immunosuppressive agents; require ongoing therapy with systemic corticosteroids in a dose higher than 10 mg prednisone per day or equivalent; are pregnant, lactating or intend to become pregnant during the study; require ongoing treatment with strong CYP1A2 inhibitors (e.g. fluvoxamine, enoxacin); and/or have a calculated creatinine clearance (or estimated glomerular filtration rate)<10 ml/min or requiring renal replacement therapy.

### Stratification Factors and Subsets

### Stratification Factors

Data is stratified according to patient hospitalization status (hospitalized/non-hospitalized), and age (less than 65 years old/65 years old and above).

### Planned Subsets

Various additional subsets are analyzed, including: age; underlying medical conditions such as lung disease, cardiovascular, immunocompromised, severe obesity (BMI 40 or above) or cancer; baseline inflammatory markers; baseline cytopenia; viral load; and concomitant medications or treatments such as anti-virals.

### Pretreatment Evaluations & Registration

### Baseline Evaluations

Required evaluations are listed in Table 2. Data for additional standard of care evaluations, such as to support eligibility criteria and subset evaluations, is collected.

### Treatment Plan

### Study Design

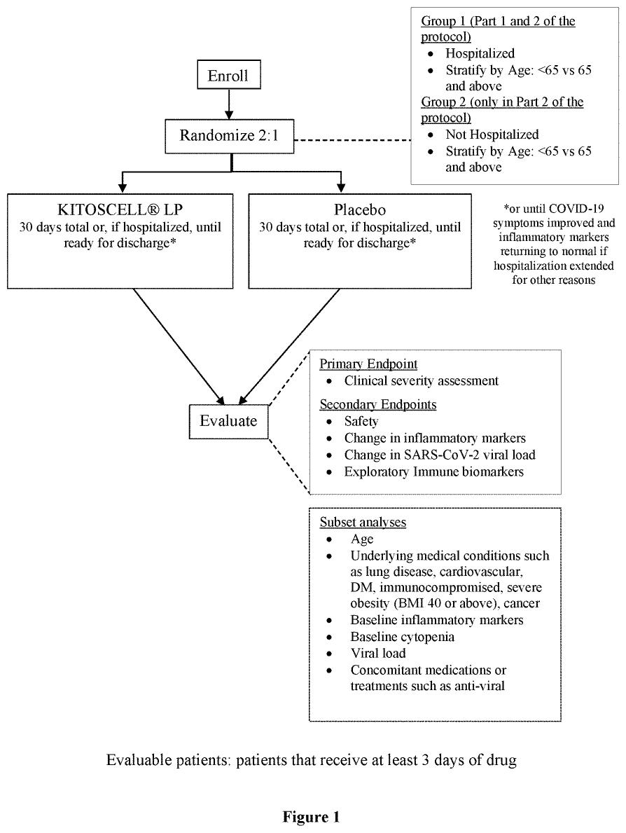

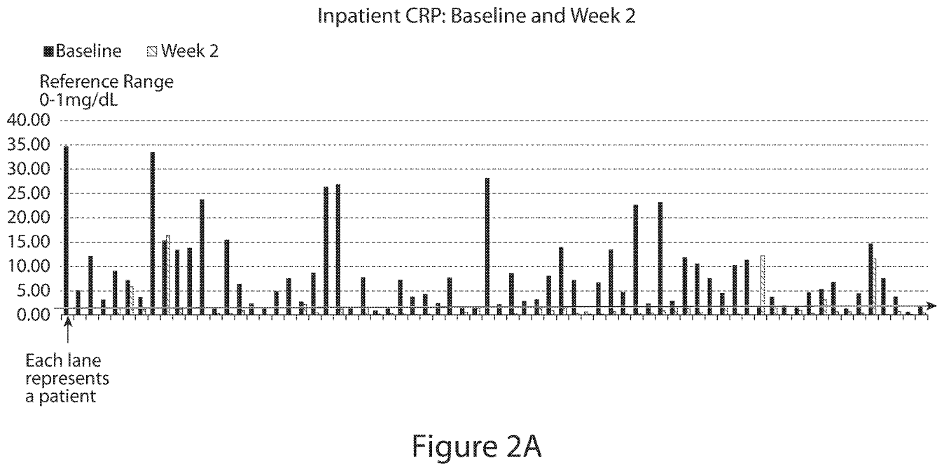

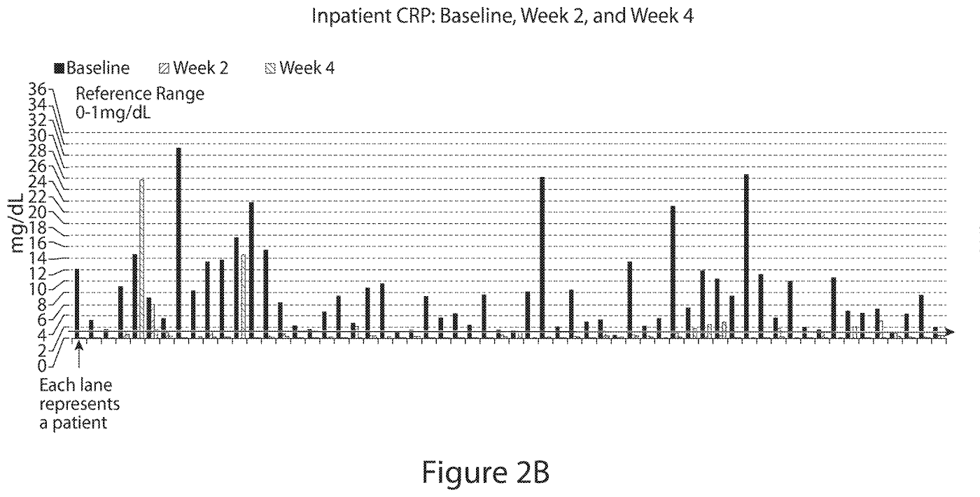

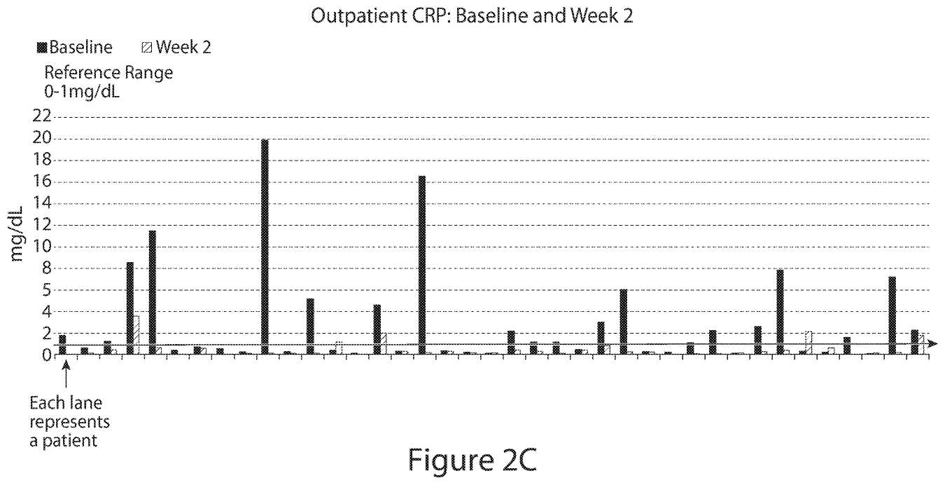

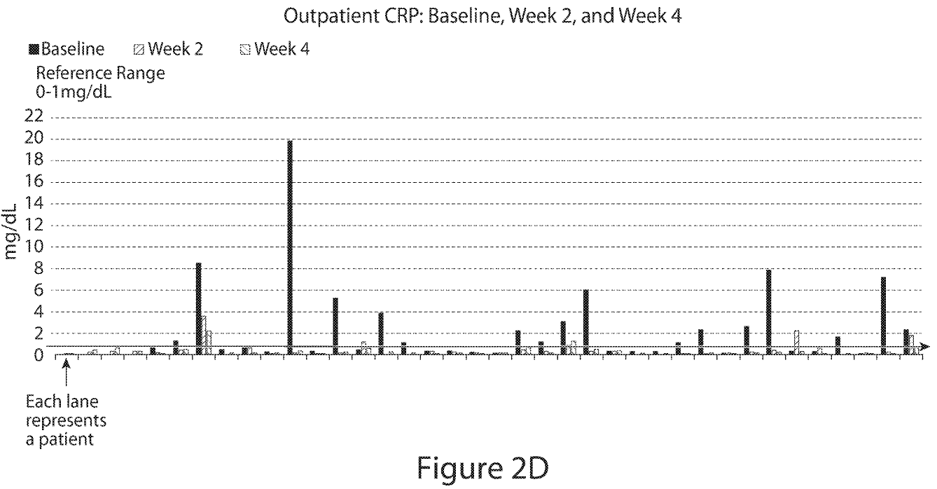

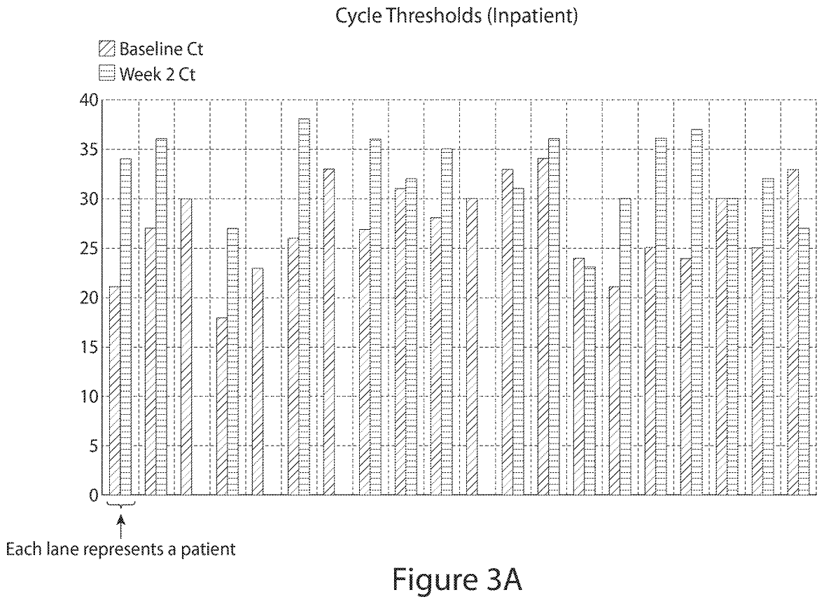

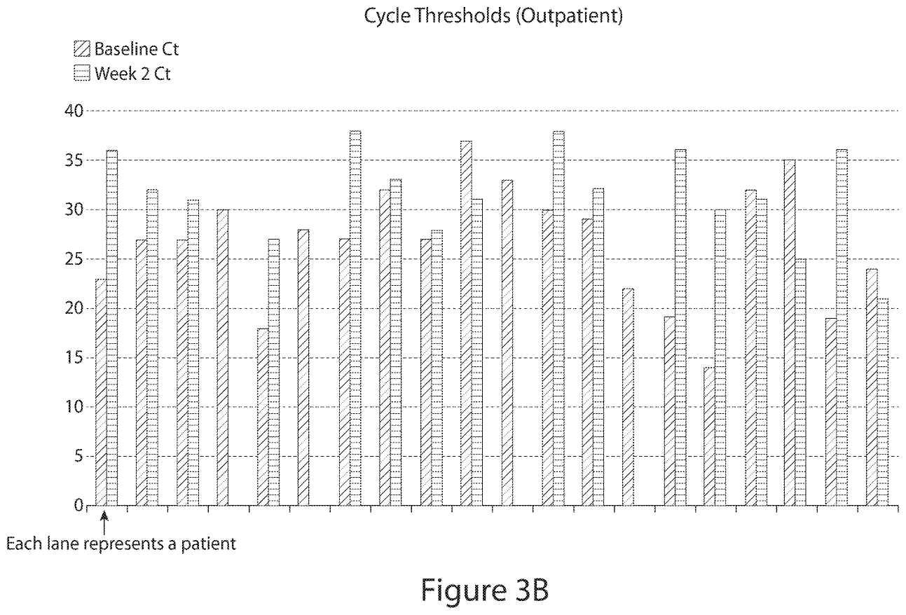

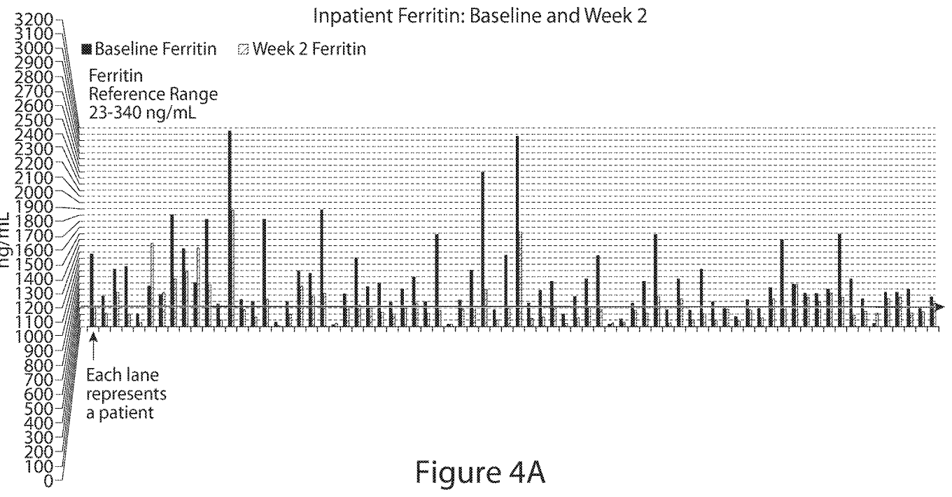

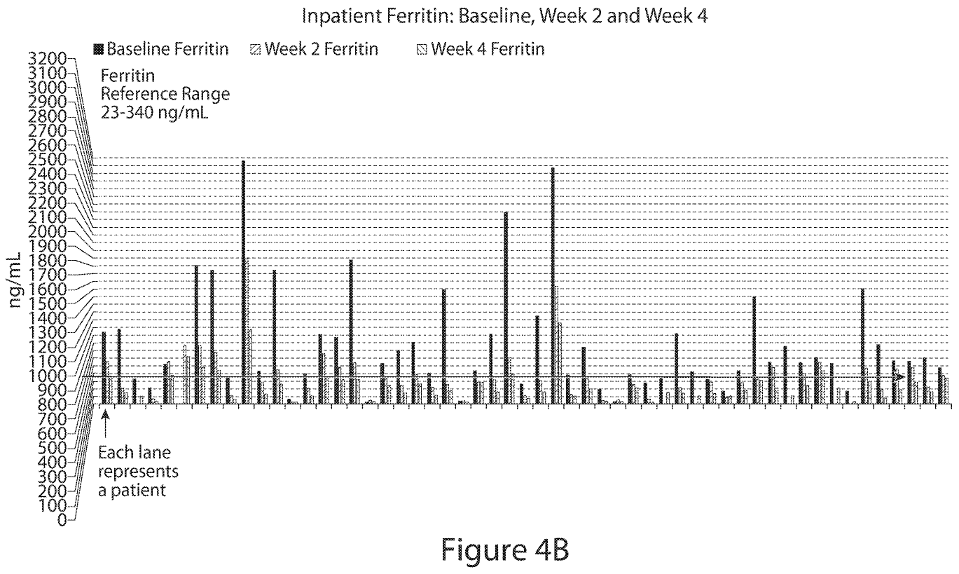

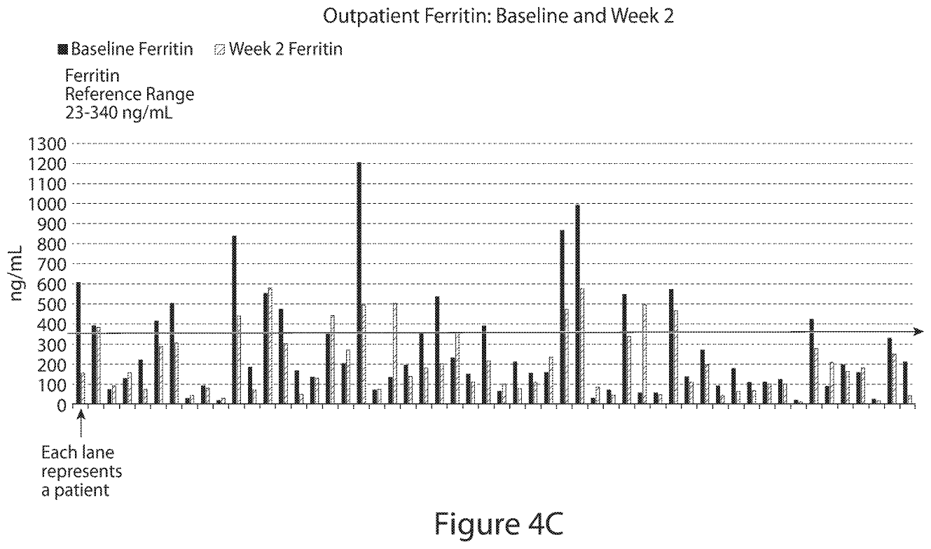

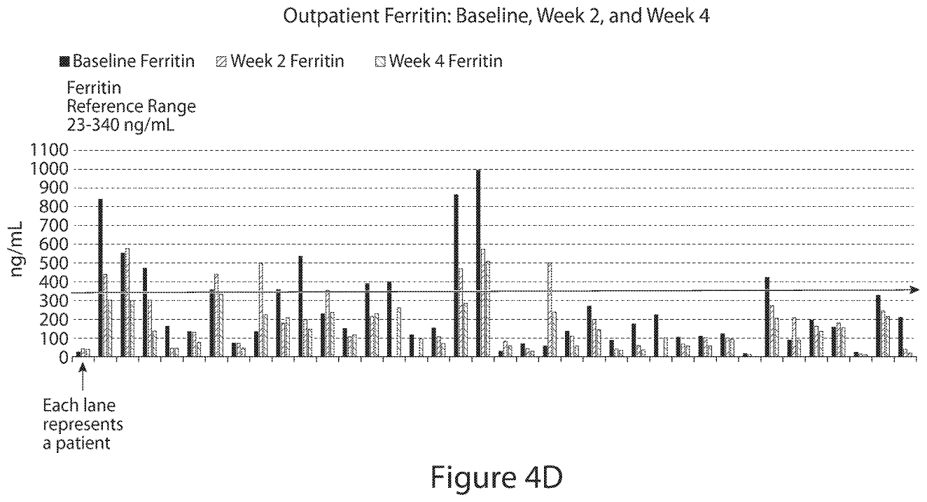

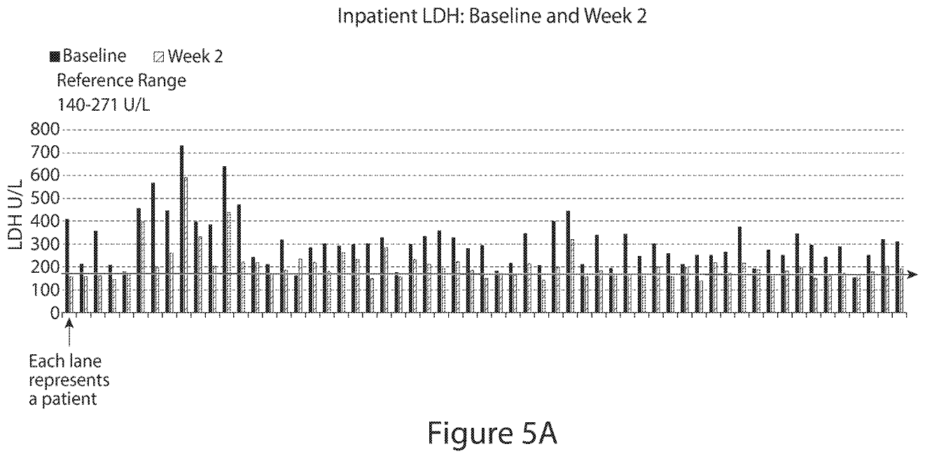

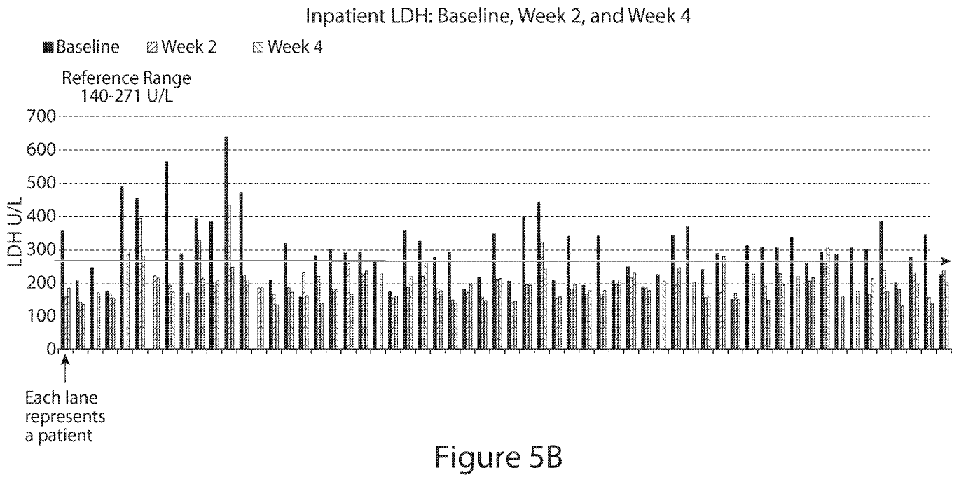

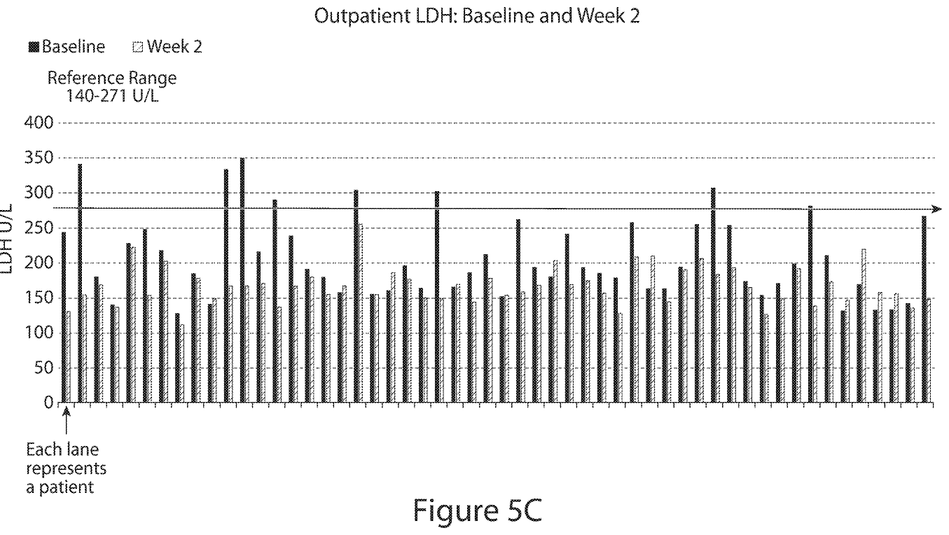

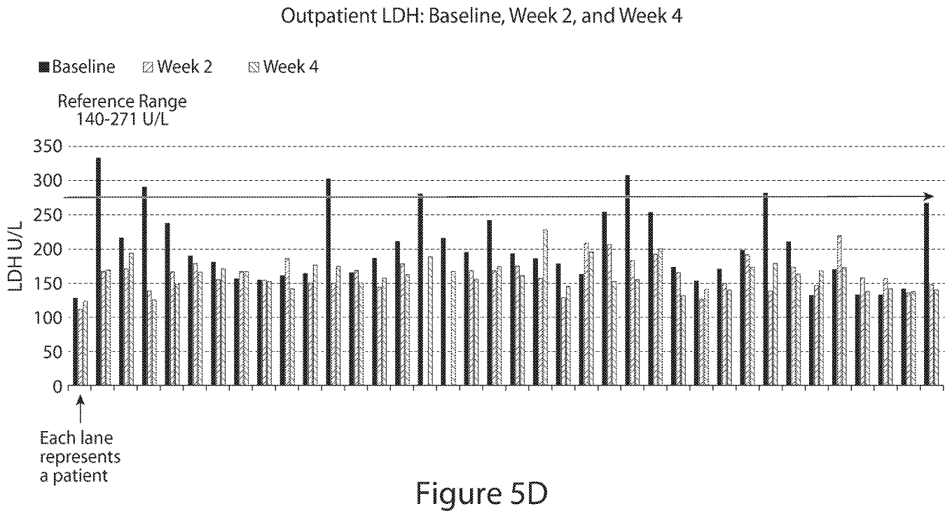

The study is a randomized, placebo-controlled blinded trial to test the safety and potential improvement in clinical outcomes for patients with COVID-19. Patients are randomized 2:1 to active and placebo arms. For treatment schedule, see FIG. 1 and Table 2, below.

### Metrics of Study:

Overall, 365 patients were enrolled (175 hospitalized and 190 ambulatory) in the trial. The group that is “hospitalized” is also referred to as “in-patient” or “inpatient,” and the group that is “ambulatory” is also referred to as “out-patient” or “outpatient.” At the Nutricion study site, 321 subjects were enrolled (131 were hospitalized and 190 ambulatory). The last patient enrolled was in November 2020. At the Citi Banamex study site, 44 were enrolled (44 were hospitalized). Of the data for FIGS. 2-5, there is data for 241 subjects (approximately 66% of the subjects in the study), for 131 hospitalized subjects and 110 ambulatory subjects, where one subject is from the Citi Banamex study site, and 240 subjects are from the Nutricion study site. The data in FIGS. 2-5 is from this population of 241 enrolled subjects (pooled, and randomized with a 2:1 ratio of active to placebo subjects).

TABLE 2
Treatment Schedule

| | | | | | | Week |
|---|---|---|---|---|---|---|
| Event | Baseline¹ | Week 1⁷ | Week 2⁷ | Week 3⁷ | Week 4⁷ | n⁸ |
| KITOSCELL ® LP or | | For 30 days or, if hospitalized, until ready for discharge* | | | | |
| placebo | | | | | | |
| SARS-CoV-2 lab test² | X | Day 7 ± 2 | Day 14 ± 2 | Day 21 ± 2 | Day 28 ± 2 | X² |
| H&P/clinical assessment | X | Inpatient daily, outpatient weekly | | | | X |
| Oxygen saturation | X | Inpatient daily, outpatient weekly | | | | X |
| Vital signs | X | Inpatient daily, outpatient weekly | | | | X |
| Co-morbidities | X | | | | | |
| Con Meds | X | Record | Record | Record | Record | |
| | | weekly | weekly | weekly | weekly | |
| Imaging as per SOC | X | If SOC | If SOC | If SOC | If SOC | If SOC |
| Labs³ | X | Day 7 ± 2 | Day 14 ± 2 | Day 21±2 | Day 28±2 | |
| Research Blood⁴ | X | Day 7 ± 2 | Day 14 ± 2 | Day 21±2 | Day 28±2 | |
| Adverse Event | | Record | Record | Record | Record | X |
| Assessment⁵ | | weekly | weekly | weekly | weekly | |
| Clinical Severity Scale⁶ | X | X | X | X | X | X |
| Drug diary with patient- | | X | X | X | X | X |
| reported symptoms | | | | | | |
| (outpatient)⁶ | | | | | | |

Table 2 Footnotes:
¹Baseline evaluations within 48 hours of starting treatment.
²SARS-CoV-2 polymerase chain reaction (PCR) or other commercial or public health assay for diagnosis and viral load. If test is pending at baseline, patient is enrolled with presumed positive based on clinical presentation and recent exposure. Patients must remain under isolation until resolution of fever, improvement in respiratory symptoms AND two consecutive negative test results are collected at least 24 hours apart.
³CBC with differential, platelets, AST, ALT, total bilirubin, creatinine, LDH, ferritin, CRP, IL-6 (if available), other labs as per standard of care (SOC)
3A. CBC with differential, platelets, AST, ALT, total bilirubin, creatinine, LDH, D-dimer, ferritin, CRP, IL-6 (if available), other labs as per SOC
⁴Blood collection for immune correlative studies
⁵Record AEs on CRF
⁶See Clinical Severity Assessment below
⁷For outpatient evaluations, visits are done by telemedicine without labs and vital signs if patient is stable and not deemed clinically necessary to have an in-person visit. Diary with patient-reported symptoms is collected, electronically as scan or photo if possible.
⁸If hospitalized, weekly monitoring is continued until discharge. If not hospitalized, one assessment (may be telemedicine) is performed 1-4 weeks after last day of study drug.
*or until COVID-19 symptoms improve and inflammatory markers return to normal if hospitalization extended for other reasons.

### Administration of KITOSCELL® LP or Placebo

### Dose

Patients are administered two pills (of KITOSCELL® LP or placebo) orally, twice per day, preferably 20-30 minutes after food. For intubated patients, one pill, crushed and made into a slurry for administration through nasogastric (NG) tube, is administered every 6 hours.

### Duration

Treatment duration for non-hospitalized patients lasted up to 30 days total. If hospitalized, patients are treated until they are ready for discharge or, if hospitalization is extended for other reasons, patients are treated until COVID-19 symptoms improve and inflammatory markers return to normal.

### Evaluable Status

Patients that receive at least three days of drug are considered evaluable.

### Criteria for Continuing Treatment

To continue treatment, a patient must have the following criteria: ALT ≤5× upper limit of normal; if ALT >3, total bilirubin must have ≤1.5× upper limit of normal, except for patients with known Gilbert disease who must have total bilirubin ≤3× upper limit of normal; if patient has renal impairment, patient is monitored for adverse events and dosage modification or discontinuation is considered.

### Concomitant Medications and Additional Treatment for COVID-19

Patients may be receiving other medications that the investigator deemed to be medically necessary. Patients may receive any other medications that the investigator deems to be standard of care or otherwise medically necessary. However, for the safety of patients and for the validity of the study disclosed herein, the following exceptions are considered. Anti-viral therapy for COVID-19 is allowed, but other investigational agents are avoided. However, anti-viral therapy for COVID-19 or rescue therapy with other immune-modulatory agents, such as an IL-6 or IL-6R antagonist, are allowed as clinically indicated with documentation of date and specific medication.

Strong CYP1A2 inhibitors (e.g., fluvoxamine, enoxacin) are not used during KITOSCELL® LP treatment. If patient deteriorated and another immune modulatory therapy is given, such as anti-IL-6 or anti-IL-6R antagonists, the date and specific medication is documented.

### Treatment Evaluation

### Evaluations During and After Treatment

Patients underwent standard of care and clinically indicated evaluations in addition to any study specific evaluations. Study specific evaluations are shown in Table 2.

### Optional Research Studies

As shown in Table 2, blood (5-15 cc) for serum for immune research studies are optionally collected in tube with no additive (e.g. standard red top tube), centrifuged (e.g. 10 minutes at 2200-2500 rpm), and the resulting serum is transferred to several sterile tubes (e.g. cryovials) for freezing. Cryovials are labeled with patient case # and date drawn. Preferably, cryovials are frozen at below −60° C.

### Toxicity Assessment

Toxicity is assessed using MedDRA coding and graded on a 5-point scale as follows: 1—mild, 2—moderate, 3—severe, 4—life-threatening, 5—death. Stopping rules for toxicity (endpoints) are described below.

### Clinical Severity Assessment

### Clinical Severity Scale

Patient status is assessed at baseline and at each post-treatment timepoint (see Table 2) using a 5-point scale and the date that a change in severity occurred is recorded. The scale is defined as follows: 1—not hospitalized; 2—hospitalized, not requiring more than 20 l/min flow; 3—hospitalized, requiring more than 201/min flow (high-flow or bipap) but not invasive mechanical ventilation; 4—hospitalized, and intubated on invasive mechanical ventilation or ECMO; 5—death.

### Patient-Reported Symptom Severity

Patients record daily in a drug diary the highest severity of fever, chills, dyspnea and cough symptoms. The patient's highest fever is to be recorded each day and is graded as follows: none [<38° C. (100.4° F.)], mild [38-39° C. (100.4-102.2° F.)], moderate [>39-40° C. (102.2 104° F.)], or severe [>40° C. (>104° F.)]. Chills, dyspnea and cough are also graded by the patient as none, mild, moderate or severe.

### Criteria for Endpoint Evaluation

The following are evaluated with additional evaluations as deemed clinically and biologically relevant: percentage of patients in each severity level post-treatment, time to stable improvement (e.g. extubated, off oxygen, and/or out of hospital), and duration of symptoms for patient-reported symptom severity. See Statistical Considerations, below for additional details.

### Secondary Endpoints

Secondary endpoints are evaluated using the following: safety based on standard laboratory and clinical adverse event monitoring; change in inflammatory markers such as serum LDH, ferritin, CRP, IL-6; change in SARS-CoV-2 viral load; and exploratory Immune biomarkers, including cytokines.

### Off Study Criteria

Patients are withdrawn from further study treatment for the following reasons: unacceptable toxicity based on treating physician assessment; patient chooses to withdraw from the study; or the investigator withdraws patient from the study for clinical judgment, such as due to lack of compliance or decision to change patient to a different treatment. Follow up for toxicity and disease outcome continues in patients withdrawn from the study for 30 days post-treatment, unless the patient withdraws from further follow up data collection.

### Study Agents—Description and Potential Risks

The KITOSCELL® LP (Pirfenidone XR) tablets contain 600 mg of pirfenidone in a sustained-release (extended release) formulation. The active ingredient, pirfenidone [1-phenyl-5-methyl-2-(1H)-pyridone], is a small synthetic, non-peptide chemical molecule, with a molecular weight of 185.2. It acts as a selective regulator of cytokines, an action that affords it specific anti-inflammatory and anti-fibrotic properties.

The most common side effects (10% or more greater than placebo) of pirfenidone are nausea, dyspepsia and rash. Due to photosensitivity, patients should avoid exposure to sunlight and sunlamps. Sunscreen and protective clothing should be worn. If a rash develops, temporary discontinuation may be necessary. AST/ALT elevation (<3%) and very rarely (0.1%) bilirubin elevation are reported with pirfenidone.

As for the placebo, Placebo tablets are physically indistinguishable from the Pirfenidone XR 600 mg tablets but contain no active ingredients. With regard to potential risks, there are no expected side effects from the placebo pills. There could be a rare intolerance to the sugars or fillers in the tablets, which contain lactose and cellulose.

### Statistical Considerations

The study disclosed herein is a randomized, placebo-controlled trial for the new, rapidly evolving, COVID-19 disease. Due to the limited information available and the evolving nature of this disease, the expected outcomes for placebo patients are estimates. The study is designed to evaluate two patient populations: 1) diagnosed hospitalized and 2) diagnosed not hospitalized. Patients are evaluable in their respective group if they take six or more drug doses prior to disease progression. Patients in Group 2 that are hospitalized prior to receiving less than six courses of drug, are evaluated with Group 1 if they complete at least six total courses of drug. Statistical review of sample size has target alpha of 0.05 and beta of 0.8. 261 patients are in Group 1 and 329 patients are in Group 2, with an interim analysis at 50% enrollment. The study is monitored by an independent clinical safety committee reviewing unblinded data at regular intervals to determine if the study should continue based on safety and potential clinical benefit. Sample size is determined using binomial superiority two-sample test of Difference of Proportions (East-5, Cytel) with an alpha of 0.05, a beta of 0.10 and a 2:1 allocation of treatment to control. For Group 1, the assumptions included a 30% failure incidence in the control group improved to a 15% in the treatment arm (hazard ratio of 0.5). With an assumption of no loss to follow-up, N=354; 236 to treatment and 118 to control. For Group 2, the assumptions included a 25% failure incidence in the control group that is improved to a 12.5% in the treatment arm (hazard ratio of 0.5). With an assumption of no loss to follow-up, N=447; 298 to treatment and 149 to control. The number of patients lost to follow-up or assessment prior to each interim analysis may be replaced by additional accrual and randomization, to a maximum of 20% of N per group. The Len-Demets spending function with O'Brien-Fleming boundaries were used for preplanned interim analysis at equal intervals of 1/3, 2/3 and final accrual of patients after completion of 30 days or termination of treatment.

### Evaluable Status of Patients

For toxicity assessment, all study-drug related toxicities from patients that received at least one dose of pirfenidone are included. Subjects receive a minimum of six doses of drug to be considered evaluable for efficacy.

### Primary Endpoint

The primary endpoint is the clinical severity assessment as described above. Patient status on the 5-point Clinical Severity Scale is determined at baseline and at each post-treatment timepoint (see Table 2) and the date that a change in severity occurred is recorded. In addition, the Patient-Reported Symptom Severity is assessed daily while patients are outpatient (see Table 2). The following are comparisons that are made using this data:

### Percentage of Patients Transitioning Between Severity Levels Post-Treatment

For evaluable patients in Group 1, the primary endpoint evaluates a difference of means between placebo and treatment for progression to mechanical ventilation or death. Early data shows 30% of placebo patients are estimated to progress to mechanical ventilation and 10% to death. A 50% decrease of progression rate of either consequence would be deemed clinically significant. In addition, the number of patients that reach disease resolution is evaluated for the two groups.

For evaluable patients in Group 2, the primary endpoint evaluates a difference of means between placebo and treatment for progression to hospitalization. Early data shows 25% of placebo patients are estimated to progress. A 50% decrease of hospitalization rate would be deemed clinically significant. In addition, the number of patients that reach each of the four levels of severity and the time to disease resolution is evaluated for the two groups.

### Time to Stable Improvement

The time in days from when a patient develops a severe event (e.g. requires hospitalization, oxygen, or intubation) to when the event resolves (e.g. discharged, off oxygen, extubated) is compared between groups.

### Duration of Symptoms for Patient-Reported Symptom Severity

Patient-Reported Symptom Severity is assessed as described above. Data is analyzed using descriptive statistics for each study group at each of the assessment points. Symptom severity between the two treatment groups is evaluated in a longitudinal fashion using the longitudinal mixed model (PROC Mixed in SAS). The model allows the change of symptom severity in the two groups over time to be investigated.

### Subset Analyses Groups

The following subsets are analyzed: age; underlying medical conditions such as lung disease, cardiovascular, immunocompromised, severe obesity (BMI 40 or above) or cancer; baseline inflammatory markers; baseline cytopenia; viral load; and concomitant medications or treatments such as anti-viral.

### Safety Evaluation

The study disclosed herein evaluates the safety of KITOSCELL® LP in patients with COVID-19. The incidence and severity (percentage of patients with grade 3 and above) of adverse events are compared between patients on placebo and KITOSCELL® LP. The proportion of toxicity events between the two groups are compared using appropriate statistical methods. Subset analyses will be performed based on age, underlying medical conditions and concomitant medications. The independent clinical safety committee evaluates the data for potential imbalance in toxicity between the treatment and control groups, based on the number of observed adverse events versus the number of patients treated. In case of significant safety concern, the study guidelines dictate that the Sponsor be alerted and the data discussed with the regulatory authorities prior to further enrollment. Overall toxicity profile analyses are based on all randomized patients who start protocol treatment according to the treatment that is actually started. An additional consideration in this trial is the potential impact of KITOSCELL® LP on viral load and duration of viral shedding. Decreasing the inflammatory response could lead to increased viral load or longer duration of shedding. Thus, the level and duration of viral shedding is compared between placebo and KITOSCELL® LP treated patients in this trial as a safety endpoint.

### Secondary Endpoints

Secondary endpoints included the following: change in inflammatory markers; change in SARS-CoV-2 viral load; and exploratory Immune biomarkers, including cytokines. Descriptive statistics and probability distribution of these endpoints are estimated using appropriate methods to generate a baseline.

### Randomization Scheme

The study uses a block stratified randomization method for treatment assignment. The randomization and treatment assignment is done centrally. The treating physician and clinical site personnel are blinded to treatment assignment.

Studies show that KITOSCELL® LP suppressed the levels of pro-inflammatory mediators that are increased in patients with COVID-19 and may contribute to the more severe symptoms of this disease. Important information is gained regarding the feasibility and potential benefit of KITOSCELL® LP with minimal risk expected for the individual patients participating in this protocol.

### Adverse Event Reporting

An adverse event (AE) is any undesirable sign, symptom or medical condition or experience that develops or worsens in severity after starting the first dose of study treatment. Study guidelines dictate that AEs are reported until 30 days after last dose of study drug.

Hospitalization, complications of hospitalization, ICU admission, intubation and death due to COVID-19 is captured in the clinical severity assessment as the primary endpoint of the study and thus would not be reported as serious adverse events (SAEs).

Additional SAE reporting guidelines are summarized in Table 3.

TABLE 1
Serious Adverse Events (SAEs) Reporting Guidelines

| | Reporting |
|---|---|
| Event | to Sponsor |
| Fatal or life-threatening | 24 hours or next |
| | business day |
| Inpatient hospitalization or prolongation | 7 days |
| of existing hospitalization | |
| A persistent or significant incapacity or | |
| substantial disruption of the ability to | |
| conduct normal life functions | |
| A congenital anomaly or birth defect. | |
| Important medical event that, based on the | |
| medical judgment of the investigator, may | |
| jeopardize the patient and may require medical | |
| or surgical intervention to prevent one of the | |
| outcomes listed above. | |

Reporting to the institutional review boards is done according to the requirements of the institution. The principal investigator or his designee is responsible for reporting to the institutional review boards and to the Sponsor.

Abnormal laboratory values or diagnostic test results constituted adverse events if they are considered clinically significant. Clinically significant values must meet one or more of the following: requires change in study medication (e.g., dose modification, interruption or permanent discontinuation) requires additional follow up (i.e., repeat labs, additional tests, etc.) result in clinical signs and/or symptoms that requires change in concomitant therapy (e.g., addition of, interruption of, discontinuation of, or any other change in a concomitant medication, therapy or treatment).

### Results for Example 1

The data in FIGS. 2-5 is from a population of 241 enrolled subjects (pooled, and randomized with a 2:1 ratio of subjects administered active drug to placebo), following administration of the active drug/placebo (randomized 2:1 ratio), at the following timepoints (baseline entry (before administration of active drug/placebo), week 2, and week 4). The CRP, ferritin, and LDH values were obtained using standard reference experimental protocols recognized by one of ordinary skill in the art. The cycle threshold (cT) levels were obtained according to the experimental protocols and methods disclosed in the following reference, incorporated herein by reference: Rao, et al., A Narrative Systematic Review of the Clinical Utility of Cycle Threshold Values in the Context of COVID-19, Infect Dis. Ther. (2020) 9:573-586.

Table 4 below is a summary of the average values of inflammatory and prognostic markers (ferritin, CRP, LDH) and cycle threshold (cT) levels (representing viral load), for the indicated number of patients (“N”), following the administration of the active drug/placebo (randomized at a 2:1 ratio). Overall, as shown in Table 4, there is a decrease in the average values of markers (ferritin, CRP, LDH) and an increase in cT (representing a decrease in viral burden), at the indicated timepoints following the administration of the active drug/placebo (randomized at a 2:1 ratio).

TABLE 4
Average Values for Inflammatory Markers and Cycle Threshold
Average Values*

| | BASELINE | | WEEK 2 | | WEEK 4 | |
|---|---|---|---|---|---|---|
| INPATIENT CRP | 7.93 | mg/dL | 0.67 | mg/dL | 1.09 | mg/dL |
| Based on a N | 60 | | 48 | | 60 | |
| OUTPATIENT CRP | 2.4 | mg/dL | 0.49 | mg/dL | 0.3 | mg/dL |
| Based on a N | 34 | | 35 | | 38 | |
| | | | | | (no recorded | |
| | | | | | baseline lab value | |
| | | | | | for 4 subjects) | |
| INPATIENT FERRITIN | 776.25 | ng/mL | 374.53 | ng/ml | 224.33 | ng/mL |
| Based on a N | 54 | | 49 | | 55 | |
| | | | | | (no recorded | |
| | | | | | baseline lab value | |
| | | | | | for 1 subject) | |
| OUTPATIENT FERRITIN | 256.48 | ng/mL | 206.02 | ng/mL | 142.55 | ng/mL |
| Based on a N | 38 | | 35 | | 38 | |
| INPATIENT LDH | 302 | U/L | 206 | U/L | 194 | U/L |
| Based on a N | 58 | | 50 | | 60 | |
| | | | | | (no recorded | |
| | | | | | baseline lab value | |
| | | | | | for 2 subjects) | |
| OUTPATIENT LDH | 203 | U/L | 163 | U/L | 161 | U/L |

| Based on a N | 38 | 36 | 38 |
|---|---|---|---|
| INPATIENT cT | 27.15 | 30.58 | |
| Based on a N | 20 | 20 | |
| OUTPATIENT cT | 26.65 | 29.7 | |
| Based on a N | 20 | 17 | |

*Average values in Table 4 are based on available data that can be compared between data at baseline, week 2 and/or week 4

Viral burden (cycle threshold) as measured by cT: cT refers to the number of cycles in reverse-transcription PCR testing required to amplify viral RNA to reach a detectable level. See Choudhuri et al., PLOS One, SARS-CoV-2 PCR cycle threshold at hospital admission associated with patient mortality, Dec. 31, 2020, //journals.plos.org/plosone/article?id=10.1371/journal.pone.0244777. An increase in Ct value correlates with a decrease in the amount of starting material (e.g., viral burden). Through data available for 20 inpatients and 20 outpatients, where the data is pooled, and randomized with a 2:1 ratio of subjects administered active drug to placebo, the data shows that the majority of these inpatients and outpatients show an increase in cT levels. See FIG. 3. Some patients showed large increases in cT levels from baseline to week 2 and beyond. See FIG. 3. Only two patients in each of the inpatient and outpatient groups had a decrease in cT levels. See FIG. 3. Overall, the data indicates that the viral burden is reduced over time in these 38 patients, out of 40 total inpatient and outpatient subjects, with some patients showing large reductions in viral burden. See FIG. 3.

Ferritin levels: Ferritin has been observed to be an important marker of severity of SARS-COV-2 disease. High levels of ferritin are associated with poorer prognosis in the SARS-COV-2 disease, including higher mortality. For this pooled data with the indicated number of subjects (randomized with a 2:1 ratio of subjects administered active drug to placebo), the data shows markedly elevated ferritin levels at the baseline, especially in some inpatients the ferritin levels are as high as 9.5× the upper limit of the reference range (23-336.2 ng/ml). See FIG. 4. In the inpatient group, the data shows that all patients had a reduction in ferritin levels, where the data for some patients showed as much as 60% reduction in ferritin levels. See FIG. 4. There were four (4) patients that showed a slight increase in ferritin levels in the outpatient group, with the majority showing a significant reduction in ferritin levels. See FIG. 4.

C-reactive protein (CRP) levels: For this pooled data with the indicated number of subjects (randomized with a 2:1 ratio of subjects administered active drug to placebo), the CRP marker of inflammation showed a similar trend of decreased levels following administration of the active drug/placebo (randomized 2:1 ratio) as for ferritin. See FIG. 2. In particular, the data shows a significant reduction in CRP levels from baseline to week 2 and week 4. See FIG. 2.

Lactic acid dehydrogenase (LDH) levels: This data for LDH levels was pooled, with the indicated number of subjects (randomized with a 2:1 ratio of subjects administered active drug to placebo). Overall, the LDH levels at baseline were much higher for the inpatient group vs the outpatient group. This is consistent with published information indicating that elevated LDH levels in COVID-19 patients are associated with marked increase in risk of severe disease, and also higher risk of mortality. There were six (6) patients in the outpatient group that had levels marginally above the reference range (140-270 U/L). The data indicates that all patients showed a marked reduction in LDH levels in both inpatient and outpatient groups, with the majority of the patients returning to normal LDH levels, following the administration of the active drug/placebo (randomized at a 2:1 ratio). Only two (2) in the inpatient group had LDH levels marginally above the upper limit of normal at follow-up timepoints (follow-up visits). Thus, the LDH data shows further internal consistency within the data in FIGS. 2-5, towards a trend to improvement in markers of inflammation in the study population.

Mortality levels: Within the population of 241 enrolled subjects, following the administration of the active drug/placebo (randomized at a 2:1 ratio), mortality data was provided for 131 inpatients, which indicated an overall lower mortality rate, 9.2% (12 of 131 inpatients), as compared to the overall mortality rate of approximately 30-60% for COVID-19 inpatients in Mexico. See Antonio Olivas-Martínez et al. PLoS One. 2021, In-hospital mortality from severe COVID-19 in a tertiary care center in Mexico City; causes of death, risk factors and the impact of hospital saturation; see Mexican Ministry of Health, Excess Mortality in Mexico, //coronavirus.gob.mx/exceso-de-mortalidad-en-mexico/, accessed Apr. 12, 2021 (indicating that actual mortality rate in Mexico is under-reported and closer to 60%); see also, Reuters, Mexico's coronavirus death toll is likely 60% higher than confirmed numbers, Mar. 29, 2021, //www.nbcnews.com/news/latino/mexicos-coronavirus-death-toll-likely-60-higher-confirmed-numbers-rcna531.

In summary, as shown in FIGS. 2-5 and in Table 4, these pooled, blinded data indicate a consistent trend in reduction of inflammatory and prognostic markers (ferritin, CRP, LDH), as well as viral burden, following the administration of the active drug/placebo (randomized at a 2:1 ratio).

### REFERENCES

1. Bhatraju, Pavan K., Bijan J. Ghassemieh, Michelle Nichols, Richard Kim, Keith R. Jerome, Arun K. Nalla, Alexander L. Greninger, et al. 2020. “Covid-19 in Critically Ill Patients in the Seattle Region Case Series.” New England Journal of Medicine, March, NEJMoa2004500. doi.org/10.1056/NEJMoa2004500.
1A. Bruss, M. L., S. B. Margolin, and S. N. Giri. 2004. “Pharmacokinetics of Orally Administered Pirfenidone in Male and Female Beagles.” Journal of Veterinary Pharmacology and Therapeutics 27 (5): 361-67. https://doi.org/10.1111/j.1365-2885.2004.00612.x.
2. Cain, W.C., R.W. Stuart, D. L. Lefkowitz, J.D. Starnes, S. Margolin, and S.S. Lefkowitz. 1998. “Inhibition of Tumor Necrosis Factor and Subsequent Endotoxin Shock by Pirfenidone.” International Journal of Immunopharmacology 20 (12): 685-95. doi.org/10.1016/S0192-0561(98)00042-3.
3. CDC COVID-19 Response Team, CDC COVID-19 Response Team, Stephanie Bialek, Ellen Boundy, Virginia Bowen, Nancy Chow, Amanda Cohn, et al. 2020. “Severe Outcomes Among Patients with Coronavirus Disease 2019 (COVID-19) United States, February 12-Mar. 16, 2020.” MMWR. Morbidity and Mortality Weekly Report 69 (12): 343-46. doi.org/10.15585/mmwr.mm6912e2.
4. Gu, Yangkui, Govindarajan Srimathveeravalli, Liqun Cai, Eisuke Ueshima, Majid Maybody, Hooman Yarmohammadi, Yuan-Shan Zhu, et al. 2018. “Pirfenidone Inhibits Cryoablation Induced Local Macrophage Infiltration along with Its Associated TGFb1 Expression and Serum Cytokine Level in a Mouse Model.” Cryobiology 82 (June): 106-11. doi.org/10.1016/j.cryobiol.2018.03.012.
5. Guo, Jingwen, Zhifeng Yang, Qiang Jia, Cunxiang Bo, Hua Shao, and Zhenling Zhang. 2019. “Pirfenidone Inhibits Epithelial-Mesenchymal Transition and Pulmonary Fibrosis in the Rat Silicosis Model.” Toxicology Letters 300 (January): 59-66. doi.org/10.1016/j.toxlet.2018.10.019.
6. Huang, Chaolin, Yeming Wang, Xingwang Li, Lili Ren, Jianping Zhao, Yi Hu, Li Zhang, et al. 2020. “Clinical Features of Patients Infected with 2019 Novel Coronavirus in Wuhan, China.” The Lancet 395 (10223): 497-506. doi.org/10.1016/S0140-6736(20)30183-5.
7. King, Talmadge E., Williamson Z. Bradford, Socorro Castro-Bernardini, Elizabeth A. Fagan, Ian Glaspole, Marilyn K. Glassberg, Eduard Gorina, et al. 2014. “A Phase 3 Trial of Pirfenidone in Patients with Idiopathic Pulmonary Fibrosis.” New England Journal of Medicine 370 (22): 2083-92. doi.org/10.1056/NEJMoa1402582.
8. Nakazato, Hitoshi, Hisashi Oku, Shoji Yamane, Yuji Tsuruta, and Ryuji Suzuki. 2002. “A Novel Anti-Fibrotic Agent Pirfenidone Suppresses Tumor Necrosis Factor-α at the Translational Level.” European Journal of Pharmacology 446 (1-3): 177-85. doi.org/10.1016/S0014-2999(02)01758-2.
9. Oku, Hisashi, Hitoshi Nakazato, Tatsuya Horikawa, Yuji Tsuruta, and Ryuji Suzuki. 2002. “Pirfenidone Suppresses Tumor Necrosis Factor-α, Enhances Interleukin-10 and Protects Mice from Endotoxic Shock.” European Journal of Pharmacology 446 (1-3): 167-76. doi.org/10.1016/S0014-2999(02)01757-0.
10. Rubino, C. M., S. M. Bhavnani, P. G. Ambrose, A. Forrest, and J. S. Loutit. 2009. “Effect of Food and Antacids on the Pharmacokinetics of Pirfenidone in Older Healthy Adults.” Pulmonary Pharmacology & Therapeutics 22 (4): 279-85. doi.org/10.1016/j.pupt.2009.03.003.
11. Schaefer, C. J., D. W. Ruhrmund, L. Pan, S. D. Seiwert, and K. Kossen. 2011. “Antifibrotic Activities of Pirfenidone in Animal Models.” European Respiratory Review 20 (120): 85-97. doi.org/10.1183/09059180.00001111.
12. Selman, Moisés, Talmadge E. King, and Annie Pardo. 2001. “Idiopathic Pulmonary Fibrosis: Prevailing and Evolving Hypotheses about Its Pathogenesis and Implications for Therapy.” Annals of Internal Medicine 134 (2): 136-51. doi.org/10.7326/0003-4819-134-2-200101160-00015.
13. Sun, Na, Isis E. Fernandez, Mian Wei, Michael Witting, Michaela Aichler, Annette Feuchtinger, Gerald Burgstaller, et al. 2018. “Pharmacometabolic Response to Pirfenidone in Pulmonary Fibrosis Detected by MALDI-FTICR-MSI.” European Respiratory Journal 52 (3): 1702314. doi.org/10.1183/13993003.02314-2017.
14. Sun, Na, Isis E. Fernandez, Mian Wei, Yin Wu, Michaela Aichler, Oliver Eickelberg, and Axel Walch. 2016. “Pharmacokinetic and Pharmacometabolomic Study of Pirfenidone in Normal Mouse Tissues Using High Mass Resolution MALDI-FTICR-Mass Spectrometry Imaging.” Histochemistry and Cell Biology 145 (2): 201-11. doi.org/10.1007/s00418-015-1382-7.
15. Wang, Zhongliang, Bohan Yang, Qianwen Li, Lu Wen, and Ruiguang Zhang. 2020. “Clinical Features of 69 Cases with Coronavirus Disease 2019 in Wuhan, China.” Clinical Infectious Diseases, March, ciaa272. doi.org/10.1093/cid/ciaa272.
16. Wu, Chaomin, Xiaoyan Chen, Yanping Cai, Jia'an Xia, Xing Zhou, Sha Xu, Hanping Huang, et al. 2020. “Risk Factors Associated With Acute Respiratory Distress Syndrome and Death in Patients With Coronavirus Disease 2019 Pneumonia in Wuhan, China.” JAMA Internal Medicine, March. doi.org/10.1001/jamainternmed.2020.0994.
17. Zhou, Fei, Ting Yu, Ronghui Du, Guohui Fan, Ying Liu, Zhibo Liu, Jie Xiang, et al. 2020. “Clinical Course and Risk Factors for Mortality of Adult Inpatients with COVID-19 in Wuhan, China: A Retrospective Cohort Study.” The Lancet 395 (10229): 1054-62. doi.org/10.1016/S0140-6736(20)30566-3.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating or preventing viremia caused by a virus in a subject in need thereof, the method comprising administering pirfenidone to the subject.

2. A method of treating or preventing viremia-induced disease in a subject in need thereof, the method comprising administering pirfenidone to the subject.

3. A method of treating COVID-19 and/or COVID-19 disease caused by a SARS-CoV-2 variant thereof in a subject in need thereof, the method comprising administering pirfenidone to the subject.

4. A method of treating or preventing an infection with a virus in a subject in need thereof, the method comprising administering pirfenidone to the subject.

5. A method of inhibiting an inflammatory response by one or more pro-inflammatory cytokines in a subject in need thereof, the method comprising administering pirfenidone to the subject.

6. A method of inhibiting a cascade by one or more pro-inflammatory cytokines in a subject in need thereof, the method comprising administering pirfenidone to the subject.

7. A method of reducing elevated levels of one or more markers of inflammation in a subject in need thereof with viremia caused by a virus, the method comprising administering pirfenidone to the subject.

8. The method of any one of claims 1-7, wherein the pirfenidone is formulated in one or more extended-release pills, wherein each of the one or more extended-release pills comprises the pirfenidone and one or more excipients.

9. The method of any one of claims 1-8, wherein each of the one or more extended-release pills comprises approximately 100 mg to approximately 850 mg of pirfenidone.

10. The method of any one of claims 1-9, wherein each of the one or more extended-release pills comprises approximately 600 mg of pirfenidone.

11. The method of any one of claims 1-10, wherein a Cₘₐₓ from a first dose of the one or more extended-release pills is between approximately 1.0 μg/mL to 10.0 μg/mL.

12. The method of any one of claims 1-11, wherein the Cₘₐₓ from a first dose of the one or more extended-release pills is between approximately 1.0 μg/mL to 4.0 μg/mL.

13. The method of any one of claims 1-7, wherein the pirfenidone is formulated in one or more sustained-release pills, wherein each of the one or more sustained-release pills comprises the pirfenidone and one or more excipients.

14. The method of any one of claims 1-8 or 13, wherein each of the one or more sustained-release pills comprises approximately 100 mg to approximately 850 mg of pirfenidone.

15. The method of any one of claims 1-9 or 14, wherein each of the one or more sustained-release pills comprises approximately 600 mg of pirfenidone.

16. The method of any one of claims 1-10 or 15, wherein a Cₘₐₓ from a first dose of the one or more sustained-release pills is between approximately 1.0 μg/mL to 10.0 μg/mL.

17. The method of any one of claims 1-11 or 16, wherein the Cₘₐₓ from a first dose of the one or more sustained-release pills is between approximately 1.0 μg/mL to 4.0 μg/mL.

18. The method of any one of claims 1-17, wherein a Tₘₐₓ is between approximately 1.5 hours to 8.0 hours.

19. The method of any one of claims 1-18, wherein the Tₘₐₓ is between approximately 3.0 hours to approximately 5.0 hours.

20. The method of any one of claims 1-19, wherein an AUC_{0-t is between approximately} 10.0 μg*hours/mL to approximately 30.0 μg*hours/mL.

21. The method of any one of claims 1-20, wherein the half-life (T_{1/2}) is between approximately 1.6 hours to approximately 8.0 hours.

22. The method of any one of claims 7-21, wherein the one or more excipients comprise one or more excipients selected from the group consisting of a compression binder, diluent, disintegrant, coating agent, rate-controlling polymer, anti-caking agent, adsorbent, lubricant, and glidant.

23. The method of any one of claims 7-22, wherein at least one excipient of the one or more excipients is selected from the group consisting of hydroxypropylmethylcellulose (HPMC), microcrystalline cellulose, silicon dioxide, and sodium stearyl fumarate.

24. The method of any one of claims 7-23, wherein each of the one or more sustained-release pills comprises:
a) approximately 100 mg to approximately 850 mg of pirfenidone;
b) microcrystalline cellulose;
c) low viscosity HPMC;
d) high viscosity HPMC;
e) silicon dioxide; and
f) sodium stearyl fumarate.

25. The method of any one of claims 7-23, wherein each of the one or more extended-release pills comprises:
a) approximately 100 mg to approximately 850 mg of pirfenidone;
b) microcrystalline cellulose;
c) low viscosity HPMC;
d) high viscosity HPMC;
e) silicon dioxide; and
f) sodium stearyl fumarate.

26. The method of any one of claims 7-23 or 25, wherein each of the one or more extended-release pills comprises:
a) approximately 100 mg to approximately 850 mg of pirfenidone;
b) approximately 100 mg to 125 mg microcrystalline cellulose;
c) approximately 50.0 mg to 100.0 mg low viscosity HPMC;
d) approximately 30.0 mg to 50.0 mg high viscosity HPMC;
e) approximately 5.0 mg to 10.0 mg silicon dioxide; and
f) approximately 5.0 mg to 10.0 mg sodium stearyl fumarate.

27. The method of any one of claims 1-24, wherein the one or more sustained-release pills are formulated as tablets, capsules, or gel capsules.

28. The method of any one of claims 1-23 or 25-27, wherein the one or more extended-release pills are formulated as tablets, capsules, or gel capsules.

29. The method of any one of claims 1-23 or 25-28, wherein the one or more extended-release pills are formulated as tablets.

30. The method of any one of claims 1, 2, or 7-29, wherein the viremia is selected from the group consisting of primary viremia, secondary viremia, active viremia, and passive viremia.

31. The method of any one of claims 1, 2, or 7-30, wherein the viremia caused by the virus is viremia caused by an RNA virus or a DNA virus.

32. The method of any one of claims 2 or 7-31, wherein the viremia-induced disease is caused by an infection with a virus.

33. The method of any one of claims 5 or 8-32, wherein the inflammatory response is induced by an infection with a virus.

34. The method of any one of claims 4 or 8-33, wherein the infection with the virus is infection with an RNA virus or a DNA virus.

35. The method of claim 34, wherein the viremia caused by an RNA virus is viremia caused by a coronavirus.

36. The method of claim 34 or 35, wherein the infection with the RNA virus is an infection with a coronavirus.

37. The method of any one of claims 34-36, wherein the coronavirus is severe acute respiratory syndrome coronavirus-2 (SARS-CoV-2).

38. The method of any one of claims 34-36, wherein the coronavirus is a SARS-CoV-2 variant of B.1.1.7, B.1.351, B.1.525, B.1.526, P.1, B.1.427, B.1.429, and/or SARS-CoV-2 variants with the Q677P and/or L452R mutations.

39. The method of any one of claims 34-38, wherein the coronavirus causes coronavirus disease 2019 (COVID-19).

40. The method of any one of claims 34-38, wherein the coronavirus is severe acute respiratory syndrome-associated coronavirus (SARS-CoV).

41. The method of any one of claims 34-38, wherein the coronavirus is Middle East respiratory syndrome coronavirus (MERS-CoV).

42. The method of any one of claims 1-41, wherein the subject develops fibrosis.

43. The method of any one of claims 1-42, wherein the fibrosis is lung fibrosis or liver fibrosis.

44. The method of any one of claims 1-43, wherein the subject has one or more contraindications for standard immediate-release pirfenidone.

45. The method of any one of claims 1-44, wherein the subject has liver disease, liver damage, or a liver injury.

46. The method of any one of claims 1-45, wherein the subject has liver disease with a Pugh-Child score of class A, class B, or class C.

47. The method of any one of claims 45 or 46, wherein the liver disease is chronic liver disease.

48. The method of any one of claims 1-47, wherein the pirfenidone is formulated in one or more extended-release tablets, the subject has one or more liver contraindications for standard-release pirfenidone; and each of the one or more extended-release tablets comprises:
a) approximately 100 mg to approximately 850 mg of pirfenidone;
b) approximately 100 mg to 125 mg microcrystalline cellulose;
c) approximately 50.0 mg to 100.0 mg low viscosity HPMC;
d) approximately 30.0 mg to 50.0 mg high viscosity HPMC;
e) approximately 5.0 mg to 10.0 mg silicon dioxide; and
f) approximately 5.0 mg to 10.0 mg sodium stearyl fumarate.

49. The method of any one of claims 1-48, comprising blocking and/or reducing an inflammatory response.

50. The method of any one of claims 1-49, comprising inhibiting the inflammatory response by one or more pro-inflammatory cytokines.

51. The method of any one of claims 1-50, wherein the inflammatory response is a cascade by the one or more pro-inflammatory cytokines.

52. The method of any one of claims 1-51, wherein the inflammatory response results in acute respiratory distress syndrome (ARDS).

53. The method of any one of claims 1-52, wherein the subject has elevated levels of one or more markers of inflammation.

54. The method of claim 53, wherein the one or more markers of inflammation are selected from the group consisting of D-dimers, C-reactive protein (CRP), ferritin, and lactic acid dehydrogenase (LDH).

55. The method of any one of claims 1-54, wherein the one or more markers of inflammation comprise C-reactive protein (CRP), ferritin, and lactic acid dehydrogenase (LDH).

56. The method of any one of claims 1-55, wherein the subject has elevated levels of one or more pro-inflammatory cytokines.

57. The method of claim 56, wherein the one or more pro-inflammatory cytokines are selected from the group consisting of TNF-α, IFN-γ, IL-1β, IL-6, and/or IL-12.

58. The method of any one of claims 1-57, wherein the subject has high viral burden and elevated levels of one or more markers of inflammation.

59. The method of any one of claims 1-58, wherein the subject that has high viral burden has low levels of cycle threshold (Ct).

60. The method of any one of claims 1-59, wherein the subject has pneumonia.

61. The method of any one of claims 1-60, wherein the subject has lung inflammation and/or lung fibrosis.

62. The method of any one of claims 1-61, wherein the subject has acute respiratory distress syndrome (ARDS).

63. The method of any one of claims 1-62, wherein the subject has COVID-19.

64. The method of any one of claims 1-63, wherein the pirfenidone is formulated in one or more extended-release pills, wherein each of the one or more extended-release pills comprises the pirfenidone and one or more excipients; the coronavirus is severe acute respiratory syndrome coronavirus-2 (SARS-CoV-2) and/or a SARS-CoV-2 variant thereof; and the subject has coronavirus disease 2019 (COVID-19) and/or a disease caused by a SARS-CoV-2 variant thereof.

65. The method of any one of claims 1-63, wherein the pirfenidone is formulated in one or more sustained-release pills, wherein each of the one or more sustained-release pills comprises the pirfenidone and one or more excipients; the coronavirus is severe acute respiratory syndrome coronavirus-2 (SARS-CoV-2); and the subject has coronavirus disease 2019 (COVID-19).

66. The method of any one of claims 1-65, wherein the subject has one or more contraindications for immediate-release pirfenidone.

67. The method of any one of claims 1-65, wherein the subject has one or more contraindications for standard-release pirfenidone.

68. The method of any one of claims 1-67, wherein a daily dosage comprises approximately 400 mg to approximately 4800 mg of pirfenidone per day.

69. The method of any one of claims 1-68, wherein a daily dosage comprises approximately 800 mg to approximately 3000 mg of pirfenidone per day.

70. The method of any one of claims 1-69, wherein a daily dosage comprises approximately 1000 mg to approximately 2400 mg of pirfenidone per day.

71. The method of any one of claims 1-70, wherein the one or more sustained-release tablets are administered orally 1 to 8 times per day.

72. The method of any one of claims 1-71, wherein the one or more sustained-release pills are administered orally two times per day.

73. The method of any one of claims 1-72, wherein the one or more sustained-release pills are administered for approximately ten days or more.

74. The method of any one of claims 1-70, wherein the one or more extended-release tablets are administered orally 1 to 8 times per day.

75. The method of any one of claims 1-70 or 74, wherein the one or more extended-release pills are administered orally two times per day.

76. The method of any one of claims 1-70 or 75, wherein the one or more extended-release pills are administered for approximately ten days or more.

77. The method of any one of claims 1-70 or 74-76, wherein the one or more extended-release pills are administered for approximately fourteen days or more.

78. The method of any one of claims 1-70 or 74-76, wherein the one or more extended-release pills are administered for approximately twenty-eight days or more.

79. The method of any one of claims 1-73, wherein the one or more sustained-release pills are administered for approximately thirty days or more.

80. The method of any one of claims 1-73 or 79, wherein the one or more sustained-release pills are administered for approximately ten to approximately thirty days.

81. The method of any one of claims 1-70 or 74-78, wherein the one or more extended-release pills are administered for approximately thirty days or more.

82. The method of any one of claims 1-70 or 74-78, wherein the one or more extended-release pills are administered for approximately ten to approximately thirty days.

83. The method of any one of claims 1-70, 74-78, 81, or 82, comprising a formulation of pirfenidone that provides release of pirfenidone over 12 hours.

84. A pharmaceutical composition comprising pirfenidone and one or more excipients, for use in treating or preventing viremia caused by a virus, viremia-induced disease, COVID-19, a disease caused by a SARS-CoV-2 variant thereof, an infection with a virus, and/or ARDS caused by infection with an infectious agent in a subject in need thereof.

85. The pharmaceutical composition of claim 84, comprising pirfenidone and one or more excipients, for use in treating or preventing viremia caused by a virus, viremia-induced disease, an infection with a virus, and/or ARDS caused by infection with an infectious agent in a subject in need thereof.

86. The pharmaceutical composition of claim 84 or 85, wherein the pirfenidone is formulated in one or more sustained-release pills, wherein each of the one or more sustained-release pills comprises the pirfenidone and the one or more excipients.

87. The pharmaceutical composition of claim 84 or 85, wherein the pirfenidone is formulated in one or more extended-release pills, wherein each of the one or more extended-release pills comprises the pirfenidone and the one or more excipients.

88. The pharmaceutical composition of claim 84-87, wherein the viremia-induced disease is caused by severe acute respiratory syndrome coronavirus-2 (SARS-CoV-2).

89. The pharmaceutical composition of claim 84-88, wherein the infection with the virus is infection with SARS-CoV-2.

90. The pharmaceutical composition of any one of claims 84-89, wherein the subject has COVID-19.

91. Use of pirfenidone for treating or preventing viremia caused by a virus, viremia-induced disease, COVID-19, a disease caused by a SARS-CoV-2 variant thereof, an infection with a virus, and/or ARDS caused by infection with an infectious agent in a subject in need thereof.

92. A kit comprising a pharmaceutical composition comprising pirfenidone and one or more excipients, for use in treating or preventing viremia caused by a virus, viremia-induced disease, COVID-19, a disease caused by a SARS-CoV-2 variant thereof, an infection with a virus, and/or ARDS caused by infection with an infectious agent; and
instructions for administering to a subject or contacting a biological sample with the pharmaceutical composition.
